SEQUENCE LISTING (79 sequences)

SEQ ID NO: 1 - DNA, 8961 bp, synthetic construct
  source 1..8961: mol_type = other DNA
  misc_feature 1..8961: note = Modified pHR'SIN:CSW vector used for lentiviral transduction and receptor expression. Receptors were inserted by In-fusion cloning (Clontech) into the BamHI (GGATCC) site downstream a PGK promoter sequence
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaattctcacgcgtcaagtggagcaaggcaggtggacagtgggatccttgacttgcggccgcaactcccacctgcaacatgcgtgactgactgaggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgaccaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctagctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggtgatcttcagacctggacgatatatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaatt

SEQ ID NO: 2 - DNA, 1626 bp, synthetic construct
  source 1..1626: mol_type = other DNA
  misc_feature 1..1626: note = Receptor GG0 sequence: Anti-CD19scFv-CD8Hinge-PolyValineTMD-Notch2JMD-Gal4VP64
gccgccaccatggccttaccagtgaccgccttgctcctgccgctggccttgctgctccacgccgccaggccggagcagaagctgatcagcgaggaggacctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgcgttgtcgttgtggtggttgtggttgttgtcgtggtggttgtggtcgtggttgtggttgtcgtcaagaggaaacggaagcacatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatcatga

SEQ ID NO: 3 - DNA, 1626 bp, synthetic construct
  source 1..1626: mol_type = other DNA
  misc_feature 1..1626: note = GG1 sequence (Anti-CD19scFv-CD8alphaHinge-PolyValin eTMD-GG1-Notch2JMD-Gal4VP64
gccgccaccatggccttaccagtgaccgccttgctcctgccgctggccttgctgctccacgccgccaggccggagcagaagctgatcagcgaggaggacctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgcggtggcgttgtggtggttgtggttgttgtcgtggtggttgtggtcgtggttgtggttgtcgtcaagaggaaacggaagcacatgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatcatga

SEQ ID NO: 4 - DNA, 63 bp, synthetic construct
  source 1..63: mol_type = other DNA
  misc_feature 1..63: note = GG2 polyvaline transmembrane domain
gttggcggtgtggtggttgtggttgttgtcgtggtggttgtggtcgtggttgtggttgtcgtc

SEQ ID NO: 5 - DNA, 63 bp, synthetic construct
  source 1..63: mol_type = other DNA
  misc_feature 1..63: note = GG3 polyvaline transmembrane domain
gttgtcggtggggtggttgtggttgttgtcgtggtggttgtggtcgtggttgtggttgtcgtc

SEQ ID NO: 6 - DNA, 63 bp, synthetic construct
  source 1..63: mol_type = other DNA
  misc_feature 1..63: note = GG4 polyvaline transmembrane domain
gttgtcgttgggggggttgtggttgttgtcgtggtggttgtggtcgtggttgtggttgtcgtc

SEQ ID NO: 7 - DNA, 63 bp, synthetic construct
  source 1..63: mol_type = other DNA
  misc_feature 1..63: note = GG5 polyvaline transmembrane domain
gttgtcgttgtggggggtgtggttgttgtcgtggtggttgtggtcgtggttgtggttgtcgtc

SEQ ID NO: 8 - DNA, 63 bp, synthetic construct
  source 1..63: mol_type = other DNA
  misc_feature 1..63: note = GG6 polyvaline transmembrane domain
gttgtcgttgtggtgggtggggttgttgtcgtggtggttgtggtcgtggttgtggttgtcgtc

SEQ ID NO: 9 - DNA, 63 bp, synthetic construct
  source 1..63: mol_type = other DNA
  misc_feature 1..63: note = GG7 polyvaline transmembrane domain
gttgtcgttgtggtggttgggggtgttgtcgtggtggttgtggtcgtggttgtggttgtcgtc

SEQ ID NO: 10 - DNA, 63 bp, synthetic construct
  source 1..63: mol_type = other DNA
  misc_feature 1..63: note = GG8 polyvaline transmembrane domain
gttgtcgttgtggtggttgtgggtggtgtcgtggtggttgtggtcgtggttgtggttgtcgtc

SEQ ID NO: 11 - DNA, 63 bp, synthetic construct
  source 1..63: mol_type = other DNA
  misc_feature 1..63: note = GG9 polyvaline transmembrane domain
gttgtcgttgtggtggttgtggttggtggcgtggtggttgtggtcgtggttgtggttgtcgtc

SEQ ID NO: 12 - DNA, 63 bp, synthetic construct
  source 1..63: mol_type = other DNA
  misc_feature 1..63: note = GG10 polyvaline transmembrane domain
gttgtcgttgtggtggttgtggttgttggcggggtggttgtggtcgtggttgtggttgtcgtc

SEQ ID NO: 13 - DNA, 63 bp, synthetic construct
  source 1..63: mol_type = other DNA
  misc_feature 1..63: note = GG11 polyvaline transmembrane domain
gttgtcgttgtggtggttgtggttgttgtcgggggggttgtggtcgtggttgtggttgtcgtc

SEQ ID NO: 14 - DNA, 63 bp, synthetic construct
  source 1..63: mol_type = other DNA
  misc_feature 1..63: note = GG12 polyvaline transmembrane domain
gttgtcgttgtggtggttgtggttgttgtcgtggggggtgtggtcgtggttgtggttgtcgtc

SEQ ID NO: 15 - DNA, 63 bp, synthetic construct
  source 1..63: mol_type = other DNA
  misc_feature 1..63: note = GG13 polyvaline transmembrane domain
gttgtcgttgtggtggttgtggttgttgtcgtggtgggtggggtcgtggttgtggttgtcgtc

SEQ ID NO: 16 - DNA, 63 bp, synthetic construct
  source 1..63: mol_type = other DNA
  misc_feature 1..63: note = GG14 polyvaline transmembrane domain
gttgtcgttgtggtggttgtggttgttgtcgtggtggttgggggcgtggttgtggttgtcgtc

SEQ ID NO: 17 - DNA, 63 bp, synthetic construct
  source 1..63: mol_type = other DNA
  misc_feature 1..63: note = GG15 polyvaline transmembrane domain
gttgtcgttgtggtggttgtggttgttgtcgtggtggttgtgggcggggttgtggttgtcgtc

SEQ ID NO: 18 - DNA, 63 bp, synthetic construct
  source 1..63: mol_type = other DNA
  misc_feature 1..63: note = GG16 polyvaline transmembrane domain
gttgtcgttgtggtggttgtggttgttgtcgtggtggttgtggtcgggggtgtggttgtcgtc

SEQ ID NO: 19 - DNA, 63 bp, synthetic construct
  source 1..63: mol_type = other DNA
  misc_feature 1..63: note = GG17 polyvaline transmembrane domain
gttgtcgttgtggtggttgtggttgttgtcgtggtggttgtggtcgtgggtggggttgtcgtc

SEQ ID NO: 20 - DNA, 63 bp, synthetic construct
  source 1..63: mol_type = other DNA
  misc_feature 1..63: note = GG18 polyvaline transmembrane domain
gttgtcgttgtggtggttgtggttgttgtcgtggtggttgtggtcgtggttgggggtgtcgtc

SEQ ID NO: 21 - DNA, 63 bp, synthetic construct
  source 1..63: mol_type = other DNA
  misc_feature 1..63: note = GG19 polyvaline transmembrane domain
gttgtcgttgtggtggttgtggttgttgtcgtggtggttgtggtcgtggttgtgggtggcgtc

SEQ ID NO: 22 - DNA, 63 bp, synthetic construct
  source 1..63: mol_type = other DNA
  misc_feature 1..63: note = GG20 polyvaline transmembrane domain
gttgtcgttgtggtggttgtggttgttgtcgtggtggttgtggtcgtggttgtggttggcggc

SEQ ID NO: 23 - skipped

SEQ ID NO: 24 - DNA, 63 bp, synthetic construct
  source 1..63: mol_type = other DNA
  misc_feature 1..63: note = CD8alpha signal peptide
atggccttaccagtgaccgccttgctcctgccgctggccttgctgctccacgccgccaggccg

SEQ ID NO: 25 - DNA, 30 bp, synthetic construct
  source 1..30: mol_type = other DNA
  misc_feature 1..30: note = myc-tag
gagcagaagctgatcagcgaggaggacctg

SEQ ID NO: 26 - DNA, 726 bp, synthetic construct
  source 1..726: mol_type = other DNA
  misc_feature 1..726: note = anti-CD19 scFv
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 27 - DNA, 63 bp, synthetic construct
  source 1..63: mol_type = other DNA
  misc_feature 1..63: note = Polyvaline transmembrane domain
gttgtcgttgtggtggttgtggttgttgtcgtggtggttgtggtcgtggttgtggttgtcgtc

SEQ ID NO: 28 - DNA, 18 bp, synthetic construct
  source 1..18: mol_type = other DNA
  misc_feature 1..18: note = Notch2 juxtamembrane domain
aagaggaaacggaagcac

SEQ ID NO: 29 - DNA, 636 bp, synthetic construct
  source 1..636: mol_type = other DNA
  misc_feature 1..636: note = Gal4-VP64
atgaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatcatga

SEQ ID NO: 30 - DNA, 63 bp, synthetic construct
  source 1..63: mol_type = other DNA
  misc_feature 1..63: note = Polyvaline transmembrane domain with GG in positions 1 and 2
ggtggcgttgtggtggttgtggttgttgtcgtggtggttgtggtcgtggttgtggttgtcgtc

SEQ ID NO: 31 - protein, 21 aa, synthetic construct
  REGION 1..21: note = CD8 alpha signal peptide
MALPVTALLLPLALLLHAARP

SEQ ID NO: 32 - protein, 10 aa, synthetic construct
  REGION 1..10: note = myc-tag
EQKLISEEDL

SEQ ID NO: 33 - DNA, 20 bp, synthetic construct
  source 1..20: mol_type = other DNA
  misc_feature 1..20: note = GAL4 DNA binding domain target sequence
ggagcactgtcctccgaacg

SEQ ID NO: 34 - protein, 21 aa, synthetic construct
  REGION 1..21: note = GG0
VVVVVVVVVVVVVVVVVVVVV

SEQ ID NO: 35 - protein, 21 aa, synthetic construct
  REGION 1..21: note = GG1
GGVVVVVVVVVVVVVVVVVVV

SEQ ID NO: 36 - protein, 21 aa, synthetic construct
  REGION 1..21: note = GG2
VGGVVVVVVVVVVVVVVVVVV

SEQ ID NO: 37 - protein, 21 aa, synthetic construct
  REGION 1..21: note = GG3
VVGGVVVVVVVVVVVVVVVVV

SEQ ID NO: 38 - protein, 21 aa, synthetic construct
  REGION 1..21: note = GG4
VVVGGVVVVVVVVVVVVVVVV

SEQ ID NO: 39 - protein, 21 aa, synthetic construct
  REGION 1..21: note = GG5
VVVVGGVVVVVVVVVVVVVVV

SEQ ID NO: 40 - protein, 21 aa, synthetic construct
  REGION 1..21: note = GG6
VVVVVGGVVVVVVVVVVVVVV

SEQ ID NO: 41 - protein, 21 aa, synthetic construct
  REGION 1..21: note = GG7
VVVVVVGGVVVVVVVVVVVVV

SEQ ID NO: 42 - protein, 21 aa, synthetic construct
  REGION 1..21: note = GG8
VVVVVVVGGVVVVVVVVVVVV

SEQ ID NO: 43 - protein, 21 aa, synthetic construct
  REGION 1..21: note = GG9
VVVVVVVVGGVVVVVVVVVVV

SEQ ID NO: 44 - protein, 21 aa, synthetic construct
  REGION 1..21: note = GG10
VVVVVVVVVGGVVVVVVVVVV

SEQ ID NO: 45 - protein, 21 aa, synthetic construct
  REGION 1..21: note = GG11
VVVVVVVVVVGGVVVVVVVVV

SEQ ID NO: 46 - protein, 21 aa, synthetic construct
  REGION 1..21: note = GG12
VVVVVVVVVVVGGVVVVVVVV

SEQ ID NO: 47 - protein, 21 aa, synthetic construct
  REGION 1..21: note = GG13
VVVVVVVVVVVVGGVVVVVVV

SEQ ID NO: 48 - protein, 21 aa, synthetic construct
  REGION 1..21: note = GG14
VVVVVVVVVVVVVGGVVVVVV

SEQ ID NO: 49 - protein, 21 aa, synthetic construct
  REGION 1..21: note = GG15
VVVVVVVVVVVVVVGGVVVVV

SEQ ID NO: 50 - protein, 21 aa, synthetic construct
  REGION 1..21: note = GG16
VVVVVVVVVVVVVVVGGVVVV

SEQ ID NO: 51 - protein, 21 aa, synthetic construct
  REGION 1..21: note = GG17
VVVVVVVVVVVVVVVVGGVVV

SEQ ID NO: 52 - protein, 21 aa, synthetic construct
  REGION 1..21: note = GG18
VVVVVVVVVVVVVVVVVGGVV

SEQ ID NO: 53 - protein, 21 aa, synthetic construct
  REGION 1..21: note = GG19
VVVVVVVVVVVVVVVVVVGGV

SEQ ID NO: 54 - protein, 21 aa, synthetic construct
  REGION 1..21: note = GG20
VVVVVVVVVVVVVVVVVVVGG

SEQ ID NO: 55 - DNA, 81 bp, synthetic construct
  source 1..81: mol_type = other DNA
  misc_feature 1..81: note = CD8alpha-derived Hinge Domain
accacgacgccagcgccgcgaccaccaacaccggcgcccaccatcgcgtcgcagcccctgtccctgcgcccagaggcgtgc

SEQ ID NO: 56 - protein, 27 aa, synthetic construct
  REGION 1..27: note = CD8alpha-derived Hinge Domain
TTTPAPRPPTPAPTIASQPLSLRPEAC

SEQ ID NO: 57 - protein, 211 aa, synthetic construct
  REGION 1..211: note = Gal4-VP64
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 58 - protein, 6 aa, synthetic construct
  REGION 1..6: note = Notch2 juxtamembrane domain
KRKRKH

SEQ ID NO: 59 - protein, 538 aa, synthetic construct
  REGION 1..538: note = Amino acid sequence corresponding to nucleotide SEQ 02 and receptor including SEQ ID 34
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVVVVVVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 60 - protein, 538 aa, synthetic construct
  REGION 1..538: note = Amino acid sequence corresponding to nucleotide SEQ 02 and receptor including SEQ ID 35
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACGGVVVVVVVVVVVVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 61 - protein, 538 aa, synthetic construct; identical to SEQ ID NO: 60
  REGION 1..538: note = Amino acid sequence corresponding to nucleotide SEQ 02 and receptor including SEQ ID 36

SEQ ID NO: 62 - protein, 538 aa, synthetic construct
  REGION 1..538: note = Amino acid sequence corresponding to nucleotide SEQ 02 and receptor including SEQ ID 37
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVGGVVVVVVVVVVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 63 - protein, 538 aa, synthetic construct
  REGION 1..538: note = Amino acid sequence corresponding to nucleotide SEQ 02 and receptor including SEQ ID 38
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVGGVVVVVVVVVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 64 - protein, 538 aa, synthetic construct
  REGION 1..538: note = Amino acid sequence corresponding to nucleotide SEQ 02 and receptor including SEQ ID 39
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVGGVVVVVVVVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 65 - protein, 538 aa, synthetic construct
  REGION 1..538: note = Amino acid sequence corresponding to nucleotide SEQ 02 and receptor including SEQ ID 40
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVGGVVVVVVVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 66 - protein, 538 aa, synthetic construct
  REGION 1..538: note = Amino acid sequence corresponding to nucleotide SEQ 02 and receptor including SEQ ID 41
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVGGVVVVVVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 67 - protein, 538 aa, synthetic construct
  REGION 1..538: note = Amino acid sequence corresponding to nucleotide SEQ 02 and receptor including SEQ ID 42
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVGGVVVVVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 68 - protein, 538 aa, synthetic construct
  REGION 1..538: note = Amino acid sequence corresponding to nucleotide SEQ 02 and receptor including SEQ ID 43
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVGGVVVVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 69 - protein, 538 aa, synthetic construct
  REGION 1..538: note = Amino acid sequence corresponding to nucleotide SEQ 02 and receptor including SEQ ID 44
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVGGVVVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 70 - protein, 538 aa, synthetic construct
  REGION 1..538: note = Amino acid sequence corresponding to nucleotide SEQ 02 and receptor including SEQ ID 45
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVVGGVVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 71 - protein, 538 aa, synthetic construct
  REGION 1..538: note = Amino acid sequence corresponding to nucleotide SEQ 02 and receptor including SEQ ID 46
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVVVGGVVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 72 - protein, 538 aa, synthetic construct
  REGION 1..538: note = Amino acid sequence corresponding to nucleotide SEQ 02 and receptor including SEQ ID 47
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVVVVGGVVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 73 - protein, 538 aa, synthetic construct
  REGION 1..538: note = Amino acid sequence corresponding to nucleotide SEQ 02 and receptor including SEQ ID 48
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVVVVVGGVVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 74 - protein, 538 aa, synthetic construct
  REGION 1..538: note = Amino acid sequence corresponding to nucleotide SEQ 02 and receptor including SEQ ID 49
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVVVVVVGGVVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 75 - protein, 538 aa, synthetic construct
  REGION 1..538: note = Amino acid sequence corresponding to nucleotide SEQ 02 and receptor including SEQ ID 50
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVVVVVVVGGVVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 76 - protein, 538 aa, synthetic construct
  REGION 1..538: note = Amino acid sequence corresponding to nucleotide SEQ 02 and receptor including SEQ ID 51
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVVVVVVVVGGVVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 77 - protein, 538 aa, synthetic construct
  REGION 1..538: note = Amino acid sequence corresponding to nucleotide SEQ 02 and receptor including SEQ ID 52
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVVVVVVVVVGGVVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 78 - protein, 538 aa, synthetic construct
  REGION 1..538: note = Amino acid sequence corresponding to nucleotide SEQ 02 and receptor including SEQ ID 53
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVVVVVVVVVVGGVKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 79 - protein, 538 aa, synthetic construct
  REGION 1..538: note = Amino acid sequence corresponding to nucleotide SEQ 02 and receptor including SEQ ID 54
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACVVVVVVVVVVVVVVVVVVVGGKRKRKHMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

(19) United States
(12) Patent Application Publication
(10) Pub. No.: US 2026/0166079 A1
(43) Pub. Date: Jun. 18, 2026

(54) NOVEL RECEPTORS WITH SYNTHETIC TRANSMEMBRANE DOMAINS FOR ENHANCED CONTROL OF LIGAND-DEPENDENT TRANSCRIPTIONAL ACTIVATION

(71) Applicant: THE REGENTS OF THE UNIVERSITY OF CALIFORNIA, Oakland, CA (US)

(72) Inventors: Kole T. ROYBAL, Oakland, CA (US); Raymond LIU, Oakland, CA (US)

(21) Appl. No.: 19/104,008

(22) PCT Filed: Aug. 14, 2023
(86) PCT No.: PCT/US2023/072180

§ 371 Date: Feb. 14, 2025

Related U.S. Application Data
(60) Provisional application No. 63/398,186, filed on Aug. 15, 2022.

(51) Int. Cl. A61K 35/14; A61K 35/30; A61K 35/36; A61K 35/44; A61K 40/42; A61P 35/02; C07K 14/47; C07K 14/705; C12N 5/071; C12N 5/0783; C12N 5/0793; C12N 15/62; C12N 15/86

(52) U.S. Cl. CPC A61K 35/14; A61K 35/30; A61K 35/36; A61K 35/44; A61K 40/42; A61P 35/02; C07K 14/4702; C07K 14/70517; C07K 14/70521; C12N 5/0619; C12N 5/0636; C12N 5/069; C12N 15/625; C12N 15/86; C07K 2317/53; C07K 2317/622; C07K 2319/02; C07K 2319/03; C12N 2830/34

(57) ABSTRACT

The present disclosure relates generally to the manipulation of chimeric polypeptide signaling to decrease cancer cell proliferation, and particularly to a new class of receptors engineered to (i) bind a target cell-surface displayed ligand and (ii) have a synthetic transmembrane domain. The disclosure also provides compositions and methods useful for producing such receptors, nucleic acids encoding same, host cells genetically modified with the nucleic acids, as well as methods for modulating an activity of a cell and/or for the treatment of various diseases such as cancers.

### CROSS REFERENCE TO RELATED APPLICATION

The present application claims priority to U.S. Provisional Patent Application Ser. No. 63/398,186 filed on Aug. 15, 2022, the disclosure of which is incorporated by reference herein in its entirety, including any drawings.

### STATEMENT REGARDING FEDERALLY SPONSORED R&D

This invention was made with government support under grant no. OD025751 awarded by The National Institutes of Health. The government has certain rights in the invention.

### INCORPORATION OF THE SEQUENCE LISTING

This application contains a Sequence Listing, which is hereby incorporated herein by reference in its entirety. The accompanying Sequence Listing file named “2023 Aug. 14 Sequence_Listing_ST26 048536-730001WO.xml” was created on Aug. 14, 2023 and is 126,933 bytes.

### FIELD

The present disclosure relates generally to a new class of receptors having synthetic transmembrane domains that bind a target cell-surface displayed ligands and to the manipulation of receptor signaling to attenuate cancer cell proliferation The disclosure also provides compositions and methods encompassing said receptors, nucleic acids encoding same, host cells genetically modified with the nucleic acids, as well as methods for modulating an activity of a cell and/or for the treatment of various diseases.

### BACKGROUND

An important problem which limits the development of gene therapy in humans is the regulation of therapeutic gene expression, such that gene expression or the vehicle used to realize expression, does not give rise to enhanced immunogenicity resulting in host rejection. One way to realize gene expression is activation of gene expression using chimeric polypeptides such as for example, Notch receptors, Notch-based receptors, or hinge-Notch receptors as disclosed in U.S. Pat. No. 11,202,801, incorporated herein in its entirety by reference.

Notch and Notch-based receptors are Type I transmembrane proteins that mediate cell-cell contact signaling and play a central role in development and other aspects of cell-to-cell communication, e.g., communication between two contacting cells, in which one contacting cell is a “receiver” cell and the other contacting cell is a “sender” cell. Notch and Notch-based receptors expressed in a receiver cell recognize their ligands (e.g., the delta/serrate/lag, or “DSL” family of proteins), expressed on a sending cell. The engagement of Notch and the DSL-ligand on these contacting cells leads to two-step proteolysis of the Notch receptor that ultimately causes the release of the intracellular portion of the receptor from the membrane into the cytoplasm. This released domain alters receiver cell behavior by functioning as a transcriptional regulator. Notch receptors are involved in and are required for a variety of cellular functions during development and are important for the function of a vast number of cell-types across species.

Notch has a metalloprotease cleavage site (denoted “S2”), which is normally protected from cleavage by the Notch negative regulatory region (NRR), which contains three LIN-12-Notch repeat (LNR) modules and a heterodimerization domain (HD) of the Notch extracellular subunit (NEC). Positioned C-terminal of the HD domain is the transmembrane domain (TMD). It contains the S3 cleavage site, which is a substrate for regulated intramembrane proteolysis by the γ-secretase complex (γSec). S3 proteolysis results in the release of the Notch intracellular domain. This event will occur only after the rate-limiting S2 cleavage has taken place, making S3 accessible to γSec

Examples of existing first-generation synthetic derivatives of Notch receptors, which are often referred to as “SynNotch receptors”, exploit this straightforward signaling behavior by replacing the extracellular ligand-binding domain, which in wild-type Notch contains multiple EGF-like repeats, with an antibody derivative, and replacing the cytoplasmic domain with a transcriptional activator of choice, while still relying on the functionality of the Notch NRR (L. Morsut et al., Cell (2016) 164:780-91). Generally, SynNotch signaling correlates with ligand binding, but it is difficult to adjust the sensitivity and response of the receptor. Additionally, the NRR spans approximately 160 amino acids, making this domain alone the size of some mature proteins, such as insulin or epidermal growth factor (EGF). This makes expression of the chimeric polypeptide less efficient and, due to vector capacity-related size constraints, the resulting chimeric polypeptides can exceed the capacity of some cloning and transfection vectors.

Next-generation SynNotch which do not require the NRR receptors are capable of binding user-defined cell surface displayed ligands and undergoing proteolytic cleavage of the receptor to release the transcriptional regulator thereby inducing a custom transcriptional program in the cell. The receptors only require a cleavable transmembrane domain, an extracellular juxtamembrane domain that can be tuned to regulate the receptor cleavage and a positively charged juxtamembrane sequence.

Receptors, whether native or synthetic, have varying characteristics, such as “noise” (i.e., the baseline level of expression induced in the absence of the intended ligand), and signal or sensitivity (the amount of expression induced by binding of the intended ligand). Generally, the signaling through Notch and “SynNotch” correlates with ligand binding, but it is difficult to adjust the sensitivity and response of the receptor, and more tools are needed in order to provide synthetic receptors with a wider range of more easily regulatable characteristics.

Another major obstacle in the efficacy of many immunotherapy-based approaches for solid tumors, including cell therapy, is delivery of drugs or activation of immune cells in the solid tumor. Cells of the monocyte/macrophage lineage make up a major component of immune cells that infiltrate into solid tumors. Because these cell types are actively recruited and retained in the solid tumor they could be an important cell type for the delivery of gene therapy.

In view of these problems, there remains a need in the art for alternative receptors that can confer enhanced control of ligand-dependent transcriptional activation and that can complement existing therapeutic standards of care for immunotherapy of cancer and other immune diseases.

### SUMMARY

The present disclosure provides, inter alia, are methods and compositions encompassing chimeric polypeptides with synthetic transmembrane domains (STMDs) that are functional in primary human T cells. Surprisingly, altering the chimeric polypeptides to encompass the STMDs still provided functional and tunable receptors that exhibit a range of signal characteristics mediated by the STMDs. These receptors, as described below, provide a range of sensitivity based on the position of particular amino acid residues in the STMD.

Provided herein are, inter alia, chimeric polypeptides having (a) an extracellular ligand-binding domain (ECD) with a binding affinity for a selected ligand, (b) a STMD including one or more ligand-inducible proteolytic cleavage site; and (c) an intracellular domain (ICD) with a transcriptional regulator (TR), wherein binding of the selected ligand to the extracellular ligand-binding domain induces cleavage at the ligand-inducible proteolytic cleavage site and release of the transcriptional regulator. In some embodiments, the chimeric polypeptides further include hinge domains located between the extracellular ligand-binding domain and the STMD. In some embodiments, the hinge domain is from CD8α or CD28. In some embodiments, the hinge domain is a truncated CD8α hinge domain or a similar hinge.

In some embodiments, the chimeric polypeptide includes the ECD, the STMD, and the ICD in order from N-terminus to C-terminus of the first polypeptide. In some embodiments, the chimeric polypeptide includes the ECD, the hinge domain, the STMD, and the ICD in order from N-terminus to C-terminus of the first polypeptide.

In some embodiments, the STMD includes one or more valine residues. In some embodiments, the STMD includes a series of at least five valine residues (i.e. five contiguous valine residues). In some embodiments, the STMD includes from 5 to 30 valine residues. In some embodiments, the STMD includes from 10 to 25 valine residues. In some embodiments, the STMD further includes two consecutive glycine residues. In some embodiments, the two consecutive glycine residues are located at any one of positions 5 to 30 of the polyvaline TMD, wherein position 30 is closer to the C-terminus of the chimeric polypeptide than position 5. In some embodiments, the STMD consists of valine residues.

In some embodiments of the chimeric polypeptides of the disclosure, the ligand includes a protein or a carbohydrate. In some embodiments, the ligand is selected from cell surface receptors, adhesion proteins, integrins, mucins, lectins, tumor associated antigens, and tumor-specific antigens. In some embodiments, the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B-cell maturation antigen (BCMA), green fluorescent protein (GFP), enhanced green fluorescent Protein (eGFP), and signal regulatory protein α (SIRPα).

In some embodiments of the chimeric polypeptides of the disclosure, the extracellular binding domain (ECD) includes the ligand-binding portion of a receptor. In some embodiments, the ECD comprises an antigen-binding moiety capable of binding, e.g., a moiety having a binding affinity to a ligand on the surface of a cell.

In some embodiments of the chimeric polypeptides of the disclosure, the antigen-binding moiety is selected from the group consisting of an antibody, a nanobody, a diabody, a triabody, or a minibody, a F(ab′) 2 fragment, a Fab fragment, a single chain variable fragment (scFv), and a single domain antibody (sdAb), or a functional fragment thereof. In some embodiments, the antigen-binding moiety comprises a scFv. In some embodiments, the antigen-binding moiety is capable of binding, e.g., a moiety having a binding affinity to a tumor-associated antigen selected from the group consisting of CD19, B7H3 (CD276), BCMA, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD 117), MUC1, NCAM, PAP, PDGFR-beta, PRSS21, PSCA, PSMA, ROR1, SSEA-4, TAG72, TEM1/CD248, TEM7R, TSHR, VEGFR2, BCMA (CD269), ALPI, citrullinated vimentin, cMet, and Axl. In some embodiments, the tumor-associated antigen is CD19, CEA, HER2, MUC1, CD20, or EGFR. In some embodiments, the tumor-associated antigen is CD19. In some embodiments, the cell is a pathogen.

In some embodiments, the ligand-inducible proteolytic cleavage site is the two consecutive glycine residues.

In some embodiments of the chimeric polypeptides, the transcriptional regulator includes a transcriptional activator, a transcriptional repressor, a site-specific nuclease, an inhibitory immunoreceptor, or an activating immunoreceptor. In some embodiments, the transcriptional regulator is selected from the group consisting of Gal4-VP16, Gal4-VP64, tetR-VP64, ZFHD1-VP64, Gal4-KRAB, and HAP1-VP16.

In some embodiments, the chimeric polypeptides of the disclosure include a ligand-induced proteolytic cleavage site, a tumor-specific cleavage site, a disease-specific cleavage site, an autoproteolytic peptide sequence, a nuclear localization signal, a juxtamembrane domain, a signaling domain, or a combination of any thereof.

In some embodiments of the chimeric polypeptide of the disclosure, the signaling domain is from DAP12, CD3 zeta, TCR zeta, FcR gamma, FcR beta, CD3 gamma, CD3 delta, CD3 epsilon, CD5, CD22, CD79a, CD79b, CD278 (ICOS), Fc.epsilon.RI, DAP10, DAP12, or CD66d.

In some embodiments, the juxtamembrane domain of the chimeric polypeptides of the disclosure is a Notch 2 juxtamembrane domain or similar polybasic domain.

In some embodiments of the chimeric polypeptides of the disclosure, the ligand-induced proteolytic cleavage site is cleavable by gamma secretase.

In some embodiments, the chimeric polypeptides of the disclosure include an autoproteolytic peptide sequence. In some embodiments, the autoproteolytic peptide sequence is from a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination of any thereof.

The chimeric polypeptide of any one of claims 1 to 31, wherein STMD includes an amino acid sequence encoded by a nucleic acid sequence having at least 80%, 85%, 90%, 95%, 96%, 97%, 98%, 99% or 100% sequence identity to any one of SEQ ID NO: 2 to SEQ ID NO: 22, SEQ ID NO: 27, or SEQ ID NO: 30.

In some embodiments of the chimeric polypeptides of the disclosure, the STMD includes an amino acid sequence having at least 80%, 85%, 90%, 95%, 96%, 97%, 98%, 99% or 100% sequence identity to any one of SEQ ID NO: 34, SEQ ID NO: 35, SEQ ID NO: 36, SEQ ID NO: 37, and SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 55.

In some embodiments, the chimeric polypeptide is encoded by a nucleic acid sequence having at least 80%, 85%, 90%, 95%, 98%, or 99% sequence identity to SEQ ID NO: 2 or SEQ ID NO: 3.

In another aspect, the disclosure provides a STMD for a chimeric polypeptide. In some embodiments, the STMD comprises at least 5 valine residues. In some embodiments, the STMD includes at least 10 valine residues. In some embodiments, the STMD includes at least 20 valine residues. In some embodiments, the STMD includes between 15 and 25 valine residues. In some embodiments, the STMD includes a ligand-inducible proteolytic cleavage site and wherein binding of the selected ligand to the extracellular ligand-binding domain induces cleavage at the ligand-inducible proteolytic cleavage site and release of the transcriptional regulator.

In a further aspect, provided herein are recombinant nucleic acid molecules including nucleotide sequences encoding the chimeric polypeptide of the disclosure

In some embodiments, the recombinant nucleic acid molecule includes a sequence having at least 80%, 85%, 90%, 95%, 98%, or 99% sequence identity to any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 27, or SEQ ID NO: 30.

In another aspect, the disclosure provides vectors including the recombinant nucleic acid molecule of the disclosure. In some embodiments, the vector is an expression vector. In some embodiments, the expression vector is a viral vector. In some embodiments, the viral vector is a lentiviral vector, an adenovirus vector, an adeno-associated virus vector, or a retroviral vector.

In another aspect, provided herein are recombinant cells including a chimeric polypeptide of the disclosure or a recombinant nucleic acid of the disclosure or a vector according to the disclosure or a STMD according to the disclosure. In some embodiments, the cell is a mammalian cell. In some embodiments, the mammalian cell is an immune cell, a neuron, an epithelial cell, and endothelial cell, or a stem cell. In some embodiments, the immune cell is a B cell, a monocyte, a natural killer cell, a basophil, an eosinophil, a neutrophil, a dendritic cell, a macrophage, a regulatory T cell, a helper T cell, a cytotoxic T cell, a CD4+ T cell, a CD8+ T cell or another T cell.

In some embodiments, the recombinant cell further includes a nucleic acid sequence encoding a protein operably linked to a promoter, wherein expression of the protein is modulated by the transcriptional regulator of the chimeric polypeptide. In some embodiments, the protein is heterologous. In some embodiments, the protein is a cytokine, a cytotoxin, a chemokine, an immunomodulator, a pro-apoptotic factor, an anti-apoptotic factor, a hormone, a differentiation factor, or a dedifferentiation factor.

In a further aspect, the disclosure provides a pharmaceutical composition comprising a recombinant cell of the disclosure.

Yet in another aspect, provided herein are methods for modulating an activity of a cell, the method including: providing a recombinant cell of the disclosure and contacting the recombinant cell with the selected ligand, wherein binding of the selected ligand to the extracellular binding domain induces cleavage of a ligand-inducible proteolytic cleavage site and releases the transcriptional regulator, wherein the released transcriptional regulator modulates an activity of the recombinant cell. In some embodiments, the contacting is carried out in vivo, ex vivo, or in vitro.

In some embodiments, wherein the activity of the cell is selected from the group consisting of: expression of a selected gene of the cell, proliferation of the cell, apoptosis of the cell, non-apoptotic death of the cell, differentiation of the cell, dedifferentiation of the cell, migration of the cell, secretion of a molecule from the cell, cellular adhesion of the cell, and cytolytic activity of the cell. In some embodiments, the released transcriptional regulator modulates expression of a gene product of the cell. In some embodiments, the released transcriptional regulator modulates expression of a heterologous gene product. In some embodiments, the gene product of the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, an RNA guided nuclease, a site-specific nuclease, a T cell receptor, a toxin, a toxin derived protein, a transcriptional activator, a transcriptional repressor, a translation regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

In some embodiments, the released transcriptional regulator modulates differentiation of the cell, and wherein the cell is an immune cell, a stem cell, a progenitor cell, or a precursor cell. In some embodiments, the administered recombinant cell modulates an activity of a target cell in the individual. In some embodiments, the target cell is a cancer cell. In some embodiments, the cancer cell is a solid tumor or a hematological malignancy cell. In some embodiments, the hematological malignancy cell is a multiple myeloma cell.

In a further aspect, the disclosure provides methods for the treatment of a health condition in an individual in need thereof, the methods include administering to the individual a first therapy comprising an effective number of the recombinant cells of the disclosure wherein the recombinant cells treat the disease in the individual. In some embodiments, the method further includes administering to the individual a second therapy. In some embodiments, the second therapy is selected from the group consisting of chemotherapy, radiotherapy, immunotherapy, hormonal therapy, and toxin therapy. In some embodiments, the first therapy and the second therapy are administered together, in the same composition or in separate compositions. In some embodiments, the first therapy and the second therapy are administered concomitantly. In some embodiments, the first therapy and the second therapy are administered sequentially. In some embodiments, the first therapy is administered before the second therapy.

In another aspect, the disclosure provides methods for inducing T cell signaling and gene regulation in a T cell including (a) providing a vector having the chimeric polypeptide of the disclosure and (b) transducing a T cell with the vector, wherein binding of a selected ligand to the extracellular ligand-binding domain of the chimeric polypeptide induces intracellular signaling and release of the transcriptional regulator.

In yet another aspect, provided herein is a system for modulating an activity of a cell, killing a target cancer cell, or treating a disease in an individual in need thereof, wherein the system includes one or more of the following: a chimeric polypeptide, a recombinant nucleic acid molecule, a recombinant cell, a pharmaceutical composition and a STMD of the disclosure.

In a further aspect, the disclosure provides a method for making the recombinant cell of the disclosure, the method includes providing a cell capable of protein expression and contacting the provided cell with a recombinant nucleic acid of the disclosure.

In another aspect, the disclosure provides use of one or more of the following for the treatment of a disease: a chimeric polypeptide, a recombinant nucleic acid molecule, a recombinant cell and a STMD of the disclosure. In some embodiments, the disease is cancer. In some embodiments, the cancer is a solid tumor or a hematological cancer. In some embodiments, the hematological cancer is multiple myeloma.

Also provided herein in an aspect, the use of any of the inventions disclosed herein for the manufacture of a medicament for the treatment of a disease.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1C illustrate a schematic comparison between a first generation SynNotch having a Notch-based regulatory region, a second generation Hinge Notch having a hinge-based regulatory region and a third generation chimeric polypeptide of the disclosure having a STMD.

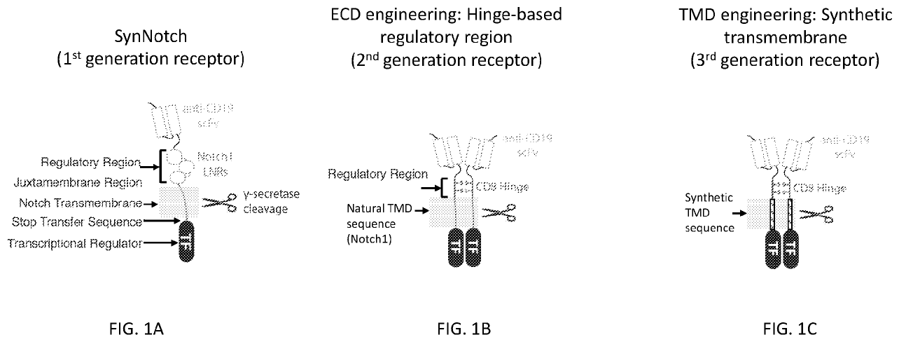

FIGS. 2A-2B schematically illustrates a non-limiting example of a chimeric polypeptide in accordance with some embodiments of the disclosure. FIG. 1A depicts the schematic structure of an exemplary chimeric polypeptide as disclosed herein (referred to as GG0 embodiment) with anti-CD19scFv extracellular domain (ECD), having a transmembrane domain made of a series of valine residues (polyvaline STMD), a Notch2 juxtamembrane domain (JMD) and an intracellular domain including a transcriptional regulator (TR) capable of regulating transcription of a BFP reporter gene. FIG. 2B schematically summarizes the results of experiments on receptor activation in primary human CD3 T cells using a BFP reporter gene. The results demonstrate that a chimeric polypeptide with a STMD and engineered to bind the B-lymphocyte antigen CD19 can activate the expression of blue-fluorescent protein BFP reporter gene when expressed in primary human CD3 T cells.

FIG. 3 illustrates structure-based engineering and activation profile of two embodiments of a chimeric polypeptide of the disclosure having a polyvaline STMD (FIG. 3A) and a modified polyvaline STMD engineered to include two destabilizing GG residues that can possibly act as proteolytic cleavage site for gamma-secretase or possibly mediate cleavage by a gamma-secretase (FIG. 3B). The reporter activation profiles for both embodiments show increased activation for the receptor engineered with GG residues in the STMD.

FIG. 4 depicts a schematic illustration of engineering strategy to test the effect of location of the destabilizing GG residues within a polyvaline STMD.

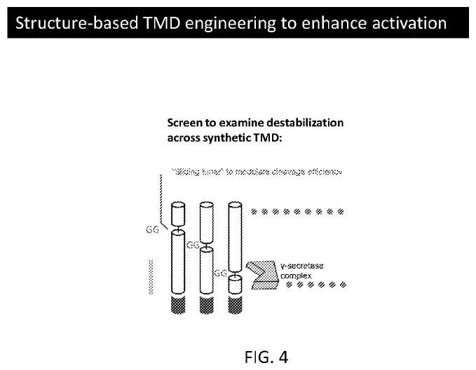

FIG. 5 schematically summaries the results of experiments performed to demonstrate the effect of placement of GG residues along the STMD towards the N terminus of the chimeric polypeptide. These experiments demonstrate that receptor activation is possible and can be tunable even with the placement of G residues towards the N terminus and away from the C terminus, in primary human CD3 T cells with K562 CD19 cells.

FIG. 6 schematically summarizes the results of activation experiments performed to demonstrate the effect of placement of GG residues along the C-terminus end of polyvaline STMD. These experiments demonstrate that receptor activation is tunable and increases as the G residues moves towards the C terminus end in primary human CD3 T cells with K562 CD19 cells.

### DETAILED DESCRIPTION OF THE DISCLOSURE

The present disclosure generally relates to a new class of chimeric polypeptides (e.g., receptors) engineered to modulate transcriptional regulation in a ligand-dependent manner while having a fully synthetic transmembrane domains (STMD). The new receptors (STMD receptors) disclosed herein do not require the Notch regulatory regions or any naturally-occurring heterologous transmembrane, previously believed to be necessary for the induced cleavage of Type I transmembrane receptors. In some embodiments, the receptors disclosed herein encompass a STMD having a series of the same residue such as, for example, a series of valines (polyvaline STMD). In some embodiments, the receptors have a GG dipeptide that may act as a proteolytic cleavage site for gamma-secretase or other proteases. Binding of a target cell-surface displayed ligand, triggers proteolytic cleavage of the receptors, possibly along the GG residues and release of a transcriptional regulator that modulates a custom transcriptional program in the cell. The disclosure also provides compositions and methods useful for producing such receptors, nucleic acids encoding same, host cells genetically modified with the nucleic acids, as well as methods for modulating an activity of a cell and/or for the treatment of various diseases such as cancers.

The receptors of the disclosure can be rationally designed with tunable features for enhanced control of proteolytic processing and possibly other intramembrane protein-protein interactions.

As described in the Examples, the new STMD receptors disclosed herein have been tested and validated in primary human T cells. One of ordinary skill in the art upon reading the disclosure will readily appreciate that the STMD receptors disclosed herein may be engineered into various immune cell types for enhanced discrimination and elimination of tumors or in engineered cells for control of autoimmunity and tissue regeneration. Accordingly, engineered cells such as immune cells engineered to express one of more of the STMD receptors disclosed herein are also within the scope of the disclosure.

The STMD receptors disclosed herein can have better activity than existing synNotches and can be a more modular platform for engineering. Existing synNotches can be engineered with ligand-binding domains such scFvs and nanobodies, but it has been difficult to use natural extracellular cellular domains from receptors/ligands on synNotches. In contrast, a number of the STMD receptors disclosed herein may be amenable to other types of ligand binding domains expanding the landscape targetable diseases and tissues.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The term “cancer” or “tumor” is used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal subject, or can be a non-tumorigenic cancer cell, such as a leukemia cell. These terms include a solid tumor, a soft tissue tumor, or a metastatic lesion. As used herein, the term “cancer” includes premalignant, as well as malignant cancers. In some embodiments, the cancer is a solid tumor, a soft tissue tumor, or a metastatic lesion.

The terms, “cell”, “cell culture”, “cell line”, “recombinant host cell”, “recipient cell”, and “host cell” as used herein, include the primary subject cells and any progeny thereof, without regard to the number of transfers.

The term “operably linked” as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. Thus, a promoter is in operable linkage with a nucleic acid sequence if it can mediate transcription of the nucleic acid sequence. It should be understood that, operably linked elements may be contiguous or non-contiguous. In the context of a polypeptide, “operably linked” refers to a physical linkage (e.g., directly or indirectly linked) between amino acid sequences (e.g., different segments, modules, or domains) to provide for a described activity of the polypeptide. In the present disclosure, various segments, region, or domains of the disclosed chimeric polypeptides and STMD receptors may be operably linked to retain proper folding, processing, targeting, expression, binding, and other functional properties of the polypeptides and receptors in the cell. Unless stated otherwise, various segments, region, or domains of the disclosed chimeric polypeptides and STMD receptors are operably linked to each other. Operably linked segments, region, or domains of the disclosed chimeric polypeptides and STMD receptors of the disclosure may be contiguous or non-contiguous (e.g., linked to one another through a linker).

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a test sequence. This definition also includes sequences that have deletions and/or additions, as well as those that have substitutions. Sequence identity typically exist over a region that is at least about 20 amino acids or nucleotides in length, or over a region that is 10-100 amino acids or nucleotides in length, or over the entire length of a given sequence.

If necessary, sequence identity can be calculated using published techniques and widely available computer programs, such as the GCS program package (Devereux et al, Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, and unless otherwise specified, a “therapeutically effective amount” of an agent is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, a “subject” or an “individual” includes animals, such as human (e.g., human subjects) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is understood that aspects and embodiments of the disclosure described herein include “comprising,” “consisting,” and “consisting essentially of” aspects and embodiments. As used herein, “comprising” is synonymous with “including,” “containing,” or “characterized by,” and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or step.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Compositions of the Disclosure

### Chimeric Polypeptides

As described in greater detail below, the present disclosure provides a new class of chimeric polypeptides engineered to modulate transcriptional regulation in a ligand-dependent manner with various advantages over existing receptors including the ability to associate additional signaling chains via charged residues.

Accordingly, the disclosure provides chimeric polypeptides including (a) an extracellular ligand-binding domain (ECD) having a binding affinity for a selected ligand; (b) a synthetic transmembrane domain (STMD) with one or more ligand-inducible proteolytic cleavage site; and (c) an intracellular domain (ICD) comprising a transcriptional regulator (TR), wherein binding of the selected ligand to the extracellular ligand-binding domain induces cleavage at the ligand-inducible proteolytic cleavage site and release of the transcriptional regulator.

### Extracellular Domains

In some embodiments, the extracellular domain of the chimeric polypeptides and the STMD receptors disclosed herein has a binding affinity for one or more target ligands. In principle, there are no particular limitations with regard to suitable ligands that may be targeted. In some embodiments, the target ligand is a cell-surface ligand. Non-limiting examples of suitable ligands include cell surface receptors, adhesion proteins, integrins, mucins, lectins. In some embodiments, the ligand is a protein. In some embodiments, the ligand is a carbohydrate.

In some embodiments, the extracellular domain of the disclosed chimeric polypeptides and STMD receptors is capable of binding, e.g., having a binding affinity to a tumor associated-antigen (TAA) or a tumor specific antigen (TSA). The term “tumor associated antigen” or “TAA” generally refers to a molecule, such as e.g., protein, present on tumor cells and on normal cells, or on many normal cells, but at much lower concentration than on tumor cells. In contrast, the term “tumor-specific antigen” or “TSA” generally refers to a molecule, such as e.g., protein which is present on tumor cells but absent from normal cells.

In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment thereof. In some embodiments, the antigen-binding moiety is selected from the group consisting of an antibody, a nanobody, a diabody, a triabody, or a minibody, a F(ab′) 2 fragment, a Fab fragment, a single chain variable fragment (scFv), and a single domain antibody (sdAb), or a functional fragment thereof. In some embodiments, the antigen-binding moiety includes a scFv.

The antigen-binding moiety can include naturally-occurring amino acid sequences or can be engineered, designed, or modified so as to provide desired and/or improved properties, e.g., binding affinity. Generally, the binding affinity of an antigen-binding moiety, e.g., an antibody, for a target antigen (e.g., CD19 antigen) can be calculated by the Scatchard method described by Frankel et al., Mol. Immunol, 16:101-106, 1979. In some embodiments, binding affinity can be measured by an antigen/antibody dissociation rate. In some embodiments, binding affinity can be measured by a competition radioimmunoassay. In some embodiments, binding affinity can be measured by ELISA. In some embodiments, antibody affinity can be measured by flow cytometry. An antibody that “selectively binds” an antigen (such as CD19) is an antigen-binding moiety that binds the antigen with high affinity and does not significantly bind other unrelated antigens.

Generally, there are no particular limitations with regard to suitable antigens that may be targeted by the chimeric polypeptides and STMD receptors disclosed herein. Non-limiting examples of suitable target antigens include CD19, B7H3 (CD276), BCMA, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRVIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD 117), MUC1, NCAM, PAP, PDGFR-beta, PRSS21, PSCA, PSMA, ROR1, SSEA-4, TAG72, TEM1/CD248, TEM7R, TSHR, VEGFR2, BCMA (CD269), ALPI, citrullinated vimentin, cMet, and Axl.

Additional antigens that can be suitable for the chimeric polypeptides and STMD receptors disclosed herein include, but are not limited to GPC2, human epidermal growth factor receptor 2 (Her2/neu), CD276 (B7-H3), IL-13-receptor alpha 1, IL-13-receptor alpha 2, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1.

Further antigens suitable for the chimeric polypeptides and STMD receptors disclosed herein include, but are not limited to the pyruvate kinase isoenzyme type M2 (tumor M2-PK), CD19, CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin. Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), and Ral-B. In some embodiments, the antigen is Glypican 2 (GPC2), CD19, human epidermal growth factor receptor 2 (Her2/neu), CD276 (B7-H3), or IL-13-receptor alpha.

In some embodiments, the chimeric polypeptides and STMD receptors disclosed herein include an extracellular domain including an antigen-binding moiety that binds CD19, CEA, HER2, MUC1, CD20, or EGFR. In some embodiments, the chimeric polypeptides and STMD receptors disclosed herein include an extracellular domain including an antigen-binding moiety that binds CD19. In some embodiments, the antigen binding moiety includes an amino acid sequence having at least 70%, at least 75%, at least 80%, at least 85%, at least 90%, at least 95%, at least 96%, at least 97%, at least 98%, at least 99% sequence identity to one or more of SEQ ID NOS: 26 in the Sequence Listing. In some embodiments, the antigen binding moiety includes an amino acid sequence having 100% sequence identity to one or more of SEQ ID NOS: 12-22 in the Sequence Listing.

In some embodiments of the chimeric polypeptides of the disclosure, the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B-cell maturation antigen (BCMA), green fluorescent protein (GFP), enhanced green fluorescent Protein (eGFP), and signal regulatory protein α (SIRPα).

In some embodiments, the ligand comprises a protein or a carbohydrate.

In some embodiments, the ligand is selected from cell surface receptors, adhesion proteins, integrins, mucins, lectins, tumor associated antigens, and tumor-specific antigens.

### Synthetic Transmembrane Domains (STMDs)

The disclosure provides STMDs and chimeric polypeptides including said STMDs. Generally, the transmembrane domain suitable for the chimeric polypeptides and STMD receptors disclosed herein can be a synthetic (STMD). In some embodiments, the STMD includes one or more alanine or leucine or valine residues or a combination thereof. In some embodiments, the STMD includes only valine residues. In some embodiments, the STMD includes one or more valine residues. In some embodiments, the STMD includes a series of at least 5 valine residues. In some embodiments, the STMD includes between 1 to 35 valine residues, between 5 to 30 valine residues, between 10 and 25 valine residues, between 15 to 20 valine residues or between 15 and 25 valine residues.

In some embodiments, the STMD includes at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 valine residues.

In some embodiments, the STMD consists of valine residues (Example GG0 encoded by SEQ ID NO: 2 in the Sequence Listing).

In some embodiments, the STMD can further include glycine residues. In some embodiments, the STMD can further include two or more glycine residues. In some embodiments, the STMD includes two consecutive glycine residues (i.e., diglycine or GG). In some embodiments, GG are located starting at any one of positions 1 to 20 or 1 to 25 or 1 to 30 of the polyvaline TMD, wherein position 30 is closer to the C-terminus of the chimeric polypeptide than position 1. In some embodiments, the GG is at position 1 (GG1), position 2 (GG2), position 3 (GG3), position 4 (GG1), position 5 (GG5), position 6 (GG6), position 7 (GG7), position 8 (GG8), position 9 (GG9), position 10 (GG10), position 11 (GG11), at position 12 (GG12), at position 13 (GG13), at position 14 (GG14), at position 15 (GG15), at position 16 (GG16), at position 17 (GG17), at position 18 (GG18), at position 19 (GG19), or at position 20 (GG20).

The STMD can include an amino acid sequence encoded by a nucleic acid sequence having at least 80%, 85%, 90%, 95%, 96%, 97%, 98%, 99%, or 100% sequence identity to any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 27, or SEQ ID NO: 30, or functional variants of any thereof.

The STMD can include an amino acid sequence having at least 80%, 85%, 90%, 95%, 96%, 97%, 98%, 99%, or 100% sequence identity to any one of SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or functional variants of any thereof.

In some embodiments, the STMD of the disclosure is GG0 encoded by SEQ ID NO: 2. In some embodiments, the STMD is any one of GG1 to GG20 receptors (SEQ ID NO: 34 to SEQ ID NO: 55. GG1 to GG20 (GGX) are the same as GG0, except with a GG substitution at the numbered location X and X+1 within the polyvaline TMD.

The STMD can include an amino acid sequence that is identical to SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or functional variants thereof.

The STMDs of the disclosure can include a ligand-inducible proteolytic cleavage site. In some embodiments, binding of the selected ligand to the extracellular ligand-binding domain of a chimeric polypeptide of the disclosure, induces cleavage at the ligand-inducible proteolytic cleavage site on the STMD and release of the transcriptional regulator.

### Linkers

Various domains of the chimeric polypeptides and STMD receptors of the disclosure can be directly fused to one another or operably linked to one another via a linker. In some embodiments, at least two of the polypeptide segments are directly linked to one another via at least one covalent bond. In some embodiments, at least two of the polypeptide segments are directly linked to one another via at least one peptide bond. In some embodiments, at least two of the polypeptide segments are operably linked to one another via a linker. There is no particular limitation on the linkers that can be used in the chimeric polypeptides described herein. In some embodiments, the linker is a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl) suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The linker can also be a linker peptide sequence. Accordingly, in some embodiments, at least two of the polypeptide segments are operably linked to one another via a linker peptide sequence. In principle, there are no particular limitations to the length and/or amino acid composition of the linker peptide sequence. In some embodiments, any arbitrary single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as a peptide linker. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker peptide sequence includes about 1 to 10, about 5 to 15, about 10 to 20, about 15 to 25, about 20 to 40, about 30 to 50, about 40 to 60, about 50 to 70 amino acid residues. In some embodiments, the linker peptide sequence includes about 40 to 70, about 50 to 80, about 60 to 80, about 70 to 90, or about 80 to 100 amino acid residues. In some embodiments, the linker peptide sequence includes about 1 to 10, about 5 to 15, about 10 to 20, about 15 to 25 amino acid residues.

### Juxtamembrane Domain

In some embodiments, the chimeric polypeptides and STMD receptors of the disclosure include a juxtamembrane domain (JMD). In these instances, the term “juxtamembrane domain” generally refers to a flexible polypeptide connector region disposed between the STMD domain and the intracellular domain (ICD) within the chimeric polypeptides and STMD receptors disclosed herein. In some embodiments, the JMD is operably linked downstream to STMD domain and upstream to the ICD domain. In principle, there are no particular limitations to the length and/or amino acid composition of the JMD. In some embodiments, any arbitrary single-chain peptide comprising about 1 to about 300 amino acid residues (e.g., 2, 3, 4, 5, 6, 7, 8, 9, 10, 11, 12, 13, 14, 15, 16, 17, 18, 19, 20, etc. amino acid residues) can be used as a JMD In some embodiments, the JMD includes about 5 to 50, about 10 to 60, about 20 to 70, about 30 to 80, about 40 to 90, about 50 to 100, about 60 to 120, about 70 to 150, about 100 to 200, about 150 to 250, about 200 to 300, about 30 to 60, about 20 to 80, about 30 to 90 amino acid residues. In some embodiments, the JMD includes about 1 to 10, about 50 to 100, about 100 to 150, about 150 to 200, about 200 to 300, about 20 to 80, about 40 to 120, about 200 to 250 amino acid residues. In some embodiments, the JMD includes about 40 to 70, about 50 to 80, about 60 to 80, about 70 to 90, or about 80 to 100 amino acid residues. In some embodiments, the JMD includes about 1 to 10, about 5 to 15, about 10 to 20, about 15 to 25 amino acid residues. In some embodiments, the JMD includes about 220, 225, 230, 235, or 240 amino acid residues. In some embodiments, the JMD includes 229 amino acid residues. In some embodiments, the length and amino acid composition of the JMD can be optimized to vary the orientation and/or proximity of the STMD domain and the ICD domain to one another to achieve a desired activity of the chimeric polypeptides and STMD receptors. In some embodiments, the orientation and/or proximity of the JMD domain and the ICD domain to one another can be varied and/or optimized as a “tuning” tool or effect that would enhance or reduce the efficacy of the chimeric polypeptides and STMD receptors.

In some embodiments, the JMD is a Notch 2 JMD. In some embodiments, the JMD includes an amino acid sequence having at least 70%, at least 75%, at least 80%, at least 85%, at least 90%, at least 95%, at least 96%, at least 97%, at least 98%, or at least 99% sequence identity to SEQ ID NO: 28 in the Sequence Listing. In some embodiments, the transmembrane domain includes an amino acid sequence having 100% sequence identity to SEQ ID NO: 28 in the Sequence Listing. In some embodiments, the JMD is a polybasic domain similar to Notch 2 JMD. In some embodiments, the polybasic domain comprises an amino acid sequence where the majority (i.e., at least 55%, at least 60%, at least 70%, at least 75%, at least 80%, at least 85%, at least 90%, at least 95%, at least 96%, at least 97%, at least 98%, at least 99%, or 100%) of the residues are lysine and/or arginine and/or histidine and/or any combination thereof.

### Hinge Domains

In some embodiments, the chimeric polypeptide or the STMD receptors of the disclosure may include a hinge domain. In these instances, the term “hinge domain” generally refers to a flexible polypeptide connector region disposed between the targeting moiety and the transmembrane domain. These sequences are generally derived from IgG subclasses (such as IgG1 and IgG4), IgD and CD8 domains, of which IgG1 has been most extensively used. In some embodiments, the hinge domain provides structural flexibility to flanking polypeptide regions. The hinge domain may consist of natural or synthetic polypeptides. It will be appreciated by those skilled in the art that hinge domains may improve the function of the chimeric polypeptides or the STMD receptors by promoting optimal positioning of the antigen-binding moiety in relationship to the portion of the antigen recognized by the same. It will be appreciated that, in some embodiments, the hinge domain may not be required for optimal chimeric polypeptide or STMD receptor activity. In some embodiments, a beneficial hinge domain comprising a short sequence of amino acids promotes the chimeric polypeptide or STMD receptor activity by facilitating antigen-binding by, e.g., relieving any steric constraints that may otherwise alter antibody binding kinetics. The sequence encoding the hinge domain may be positioned between the antigen recognition moiety and the transmembrane domain. In some embodiments, the hinge domain is operably linked downstream of the antigen-binding moiety and upstream of the transmembrane domain.

In some embodiments, the Hinge Domain is CD8α hinge domain. In some embodiments, the hinge domain is a truncatedCD8α hinge domain. In some embodiments, the CD8α hinge domain includes an amino acid sequence having at least 70%, at least 75%, at least 80%, at least 85%, at least 90%, at least 95%, at least 96%, at least 97%, at least 98%, or at least 99% sequence identity to SEQ ID NO: 56 in the Sequence Listing. In some embodiments, the transmembrane domain includes an amino acid sequence having 100% sequence identity to SEQ ID NO: 56 in the Sequence Listing. In some embodiments, the truncated CD8α hinge domain is encoded by a nucleotide sequence having at least 80%, 85%, 90%, 95%, 96%, 97%, 98%, 99%, or 100%, or any value in between, sequence identity to SEQ ID NO: 55, or functional variants thereof.

### Intracellular Domain

In some embodiments, the chimeric polypeptide or the STMD receptors of the disclosure include an intracellular domain (ICD). The intracellular domain can have an intracellular biological activity. The ICD can include a transcriptional regulator. The transcriptional regulator of the disclosure is a polypeptide element that acts to activate or inhibit the transcription of a promoter-driven DNA sequence. Transcriptional regulators suitable for the compositions and methods of the disclosure can be naturally-occurring transcriptional regulators or can be engineered, designed, or modified so as to provide desired and/or improved properties, e.g., modulating transcription. As discussed above, the engineered receptors of the present disclosure are advantageous in that they can provide the ability to trigger a custom transcriptional program in engineered cells. In some embodiments, transcriptional regulator of the disclosure is a custom transcriptional regulator that drives transcription off a specific sequence that only appears once in the engineered cell.

In some embodiments, the transcriptional regulator directly regulates differentiation of the cell. In some embodiments, the transcriptional regulator indirectly modulates (e.g., regulates) differentiation of the cell by modulating the expression of a second transcription factor. It will be understood by one having ordinary skill in the art that a transcriptional regulator can be a transcriptional activator or a transcriptional repressor. In some embodiments, the transcriptional regulator is a transcriptional repressor. In some embodiments, the transcriptional regulator is a transcriptional activator. In some embodiments, the transcriptional regulator can further include a nuclear localization signal. In some embodiments, the transcriptional regulator is selected from Gal4-VP16, Gal4-VP64, tetR-VP64, ZFHD1-VP64, Gal4-KRAB, and HAP1-VP16. In some embodiments, the transcriptional regulator is Gal4-VP64.

Chimeric polypeptides and STMD receptors of the present disclosure can be chimeric polypeptides of any length, including chimeric polypeptides that are generally between about 100 amino acids (aa) to about 1000 aa, e.g., from about 100 aa to about 200 aa, from about 150 aa to about 250 aa, from about 200 aa to about 300 aa, from about 250 aa to about 350 aa, from about 300 aa to about 400 aa, from about 350 aa to about 450 aa, from about 400 aa to about 500 aa in length. In some embodiments, the disclosed chimeric polypeptides are generally between about 400 aa to about 450 aa, from about 450 aa to about 500 aa, from about 500 aa to about 550 aa, from about 550 aa to about 600 aa, from about 600 aa to about 650 aa, from about 650 aa to about 700 aa, from about 700 aa to about 750 aa, from about 750 aa to about 800 aa, from about 800 aa to about 850 aa, from about 850 aa to about 900 aa, from about 900 aa to about 950 aa, or from about 950 aa to about 1000 aa in length. In some cases, the chimeric polypeptides of the present disclosure have a length of about 300 aa to about 400 aa. In some cases, the chimeric polypeptides of the present disclosure have a length of about 300 aa to about 350 aa. In some cases, the chimeric polypeptides of the present disclosure have a length of about 300 aa to about 325 aa. In some cases, the chimeric polypeptides of the present disclosure have a length of about 350 aa to about 400 aa. In some cases, the chimeric polypeptides of the present disclosure have a length of 750 aa to 850 aa. In some embodiments, the chimeric polypeptides of the present disclosure have a length of about 525 aa, about 538 aa, about 539 aa, about 542 aa, about 550 aa, about 556 aa, or about 697 aa.

### Nucleic Acids

In one aspect, some embodiments disclosed herein relate to nucleic acid molecules that include nucleotide sequences encoding the chimeric polypeptides and STMD receptors of the disclosure, including expression cassettes, and expression vectors containing these nucleic acid molecules operably linked to heterologous nucleic acid sequences.

The disclosure includes compositions and methods for introduction of chimeric polypeptide system components into cells. Introduction of nucleic acids into cells may be done in a number of ways, including by methods described in many standard laboratory manuals, such as Davis et al., Basic methods in molecular biology, (1986); Sambrook et al., Molecular cloning: A laboratory manual 2nd Ed., Cold Spring Harbour Laboratory Press, Cold Spring Harbour, N.Y. (1989); and Kim and Eberwine, Mammalian cell transfection: the present and the future (2010), such as calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

The disclosure includes methods in which different chimeric polypeptides or receptor components are introduced into cells by different means, as well as compositions of matter for performing such methods. For example, a lentiviral vector may be used to introduce chimeric polypeptide system component-coding nucleic acid by transfection.

In most instances, the chimeric polypeptides or receptor components will be introduced into a cell in a manner that results in the generation of the chimeric polypeptide by the cell. Thus, in instances where a cell expresses a chimeric polypeptide protein, the cell had been transfected with a chimeric polypeptide-encoding nucleic acid operably linked to a promoter, which has then been, e.g., chromosomally integrated.

Transfection agents suitable for use with the disclosure include transfection agents that facilitate the introduction of RNA, DNA, and proteins into cells. Exemplary transfection reagents include TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LcIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.).

The disclosure further includes methods in which one molecule is introduced into a cell, followed by the introduction of another molecule into the cell. Thus, more than one chimeric polypeptide or receptor component may be introduced into a cell at the same time or at different times. As an example, the disclosure includes methods in which a chimeric polypeptide or STMD receptor encoding nucleic acid is introduced into a cell while the cell is in contact with a transfection reagent designed to facilitate the introduction of nucleic acids into cells (e.g., TurboFect Transfection Reagent), followed by washing the cells and then introducing another chimeric polypeptide component while the cell is in contact with, e.g., LIPOFECTAMINE™ 2000.

Additionally and/or alternatively, introduction of nucleic acids into cells may be achieved using viral transduction methods. Transduction does not require physical contact between the cell donating the DNA and the cell receiving the DNA, and it is DNase resistant. Adeno-associated virus (AAV) is a non-enveloped virus that can be engineered to deliver nucleic acids to target cells via viral transduction. The ability to generate AAV particles lacking any viral genes and containing nucleic acid sequences of interest for various therapeutic applications has thus far proven to be one of the safest strategies for gene therapy. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses

Lentiviral systems are also amenable for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) potentially safer integration site profile; and (vii) a relatively easy system for vector manipulation and production.

As an example, the disclosure includes methods in which a chimeric polypeptide-encoding nucleic acid is introduced into a cell using an expression cassette or expression vector, such as a viral vector. The viral vector may be produced according to methods presented in the art, such as in Watson and Wolfe et al. Transduction of the target cells can be carried out with the desired cell numbers and multiplicities of infections (MOIs) of the vector for approx. 2 hours in suitable media. Cells may be prepared so that they grow exponentially and are no more than 70-80% confluent before transduction. Cells may be added in fresh medium to the wells of 96-well plate followed by incubation at 37° C. in a humidified incubator. Lentiviral vectors may be added to appropriate wells, gently mixed and incubated at 37° C. in a humidified incubator. Due to toxicity concerns of lentiviral vectors, cells may be incubated for 2 to 4 hours before changing the medium containing lentiviral vectors (Nasri et al.)

Disclosed herein are nucleic acid molecules encoding the chimeric polypeptide system components of the disclosure, expression cassettes, and expression vectors containing these nucleic acid molecules operably linked to regulator sequences which allow expression of the chimeric polypeptide system components in a host cell or ex-vivo cell-free expression system.

The terms “nucleic acid molecule” and “polynucleotide” are used interchangeably herein, and refer to both RNA and DNA molecules, including nucleic acid molecules including cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

Nucleic acid molecules of the present disclosure can be of any length, and are generally between about 5 Kb and about 50 Kb, for example between about 5 Kb and about 40 Kb, between about 5 Kb and about 30 Kb, between about 5 Kb and about 20 Kb, or between about 10 Kb and about 50 Kb, about 15 Kb to about 30 Kb, about 20 Kb to about 50 Kb, about 20 Kb to about 40 Kb, about 5 Kb to about 25 Kb, or about 30 Kb to about 50 Kb.

In some embodiments, the nucleic acid molecules encode a polypeptide with an amino acid sequence having at least about 80%, 90%, 95%, 96%, 97, 98%, 99%, or 100% sequence identity to the first or the second polypeptide chain of a chimeric polypeptide as disclosed herein. In some embodiments, the nucleic acid molecules encode a single chain polypeptide with an amino acid sequence having at least about 80%, 90%, 95%, 96%, 97, 98%, 99%, or 100% sequence identity to any one of the amino acid sequences identified in the Sequence Listing. In some embodiments, the nucleic acid molecules encode a polypeptide with an amino acid sequence having at least about 80%, 90%, 95%, 96%, 97, 98%, 99%, or 100% sequence identity to any one of the chimeric polypeptide amino acid sequences identified in the Sequence Listing. In some embodiments, the nucleic acid molecules of the disclosure encode a single chain polypeptide with an amino acid sequence having at least about 80%, 90%, 95%, 96%, 97, 98%, 99%, or 100% sequence identity to any one of SEQ ID NO: 59 to SEQ ID NO: 79.

Some embodiments disclosed herein relate to vectors or expression cassettes including a recombinant nucleic acid molecule encoding the chimeric polypeptides disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into a subject. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any chimeric polypeptide disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. Additional vectors can also be found, for example, in Ausubel, F. M., et al., Current Protocols in Molecular Biology, New York, NY: Wiley (including supplements through 2014), and Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001).

In some embodiments, the chimeric polypeptide components are expressed from vectors, such as expression vectors. The vectors are useful for autonomous replication in a host cell, or may be integrated into the genome of a host cell, and thereby are replicated along with the host genome (e.g., non-episomal mammalian vectors). Expression vectors are capable of directing the expression of coding sequences to which they are operably linked. In general, expression vectors are often in the form of plasmids. However, other forms of expression vectors, such as viral vectors (e.g., replication defective retroviruses, adenoviruses, and adeno-associated viruses) are also included. Exemplary recombinant expression vectors can include one or more regulatory sequences, selected on the basis of the host cells to be used for expression, operably linked to the nucleic acid sequence to be expressed.

DNA vectors can be introduced into prokaryotic or eukaryotic cells via conventional transformation or transfection techniques. Suitable methods for transforming or transfecting host cells can be found in Sambrook et al. (2001, supra) and other standard molecular biology laboratory manuals.

The nucleic acid sequences encoding the chimeric polypeptides can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the chimeric polypeptide disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Vectors suitable for use include T7-based vectors for use in bacteria, the pMSXND expression vector for use in mammalian cells, and baculovirus-derived vectors for use in insect cells.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Viral vectors can include a modified pHR′ SIN:CSW vector used for lentiviral transduction and receptor expression (SEQ ID NO: 1). The chimeric polypeptides of the disclosure can be inserted by In-fusion cloning (Clontech) into the BamHI (GGATCC) site downstream a PGK promoter sequence.

The precise components of the expression system are not critical. For example, a chimeric polypeptide as disclosed herein can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed antibody can be purified from the expression system using routine biochemical procedures, and can be used, e.g., as therapeutic agents, as described herein.

In some embodiments, chimeric polypeptides or STMD receptors obtained will be glycosylated or unglycosylated depending on the host organism used to produce the chimeric polypeptides. If bacteria are chosen as the host then the chimeric polypeptides produced will be unglycosylated. Eukaryotic cells, on the other hand, will glycosylate the chimeric polypeptides, although perhaps not in the same way as native polypeptides is glycosylated. The recombinant antibodies produced by the transformed host can be purified according to any suitable methods known in the art. Produced recombinant antibodies can be isolated from inclusion bodies generated in bacteria such as E. coli, or from conditioned medium from either mammalian or yeast cultures producing a chimeric polypeptide of the disclosure using cation exchange, gel filtration, and or reverse phase liquid chromatography.

In addition or alternatively, another exemplary method of constructing a DNA sequence encoding the chimeric polypeptides of the disclosure is by chemical synthesis. This includes direct synthesis of a peptide by chemical means of the amino acid sequence encoding for a chimeric polypeptide exhibiting the properties described. This method can incorporate both natural and unnatural amino acids at positions that affect the binding affinity of the chimeric polypeptides with a target protein. Alternatively, a gene which encodes the desired chimeric polypeptides can be synthesized by chemical means using an oligonucleotide synthesizer. Such oligonucleotides are designed based on the amino acid sequence of the desired chimeric polypeptides, and preferably selecting those codons that are favored in the host cell in which the chimeric polypeptide of the disclosure will be produced. In this regard, it is well recognized in the art that the genetic code is degenerate such that an amino acid may be coded for by more than one codon. For example, Phe (F) is coded for by two codons, TIC or TTT, Tyr (Y) is coded for by TAC or TAT and his (H) is coded for by CAC or CAT. Trp (W) is coded for by a single codon, TGG. Accordingly, it will be appreciated by those skilled in the art that for a given DNA sequence encoding a particular chimeric polypeptide, there will be many DNA degenerate sequences that will code for that chimeric polypeptide. For example, it will be appreciated that in addition to the DNA sequences for chimeric polypeptides provided herein, there will be many degenerate DNA sequences that code for the chimeric polypeptides disclosed herein. These degenerate DNA sequences are considered within the scope of this disclosure. Therefore, “degenerate variants thereof” in the context of this disclosure means all DNA sequences that code for and thereby enable expression of a particular chimeric polypeptide.

The DNA sequence encoding the subject chimeric polypeptide, whether prepared by site directed mutagenesis, chemical synthesis or other methods, can also include DNA sequences that encode a signal sequence. Such signal sequence, if present, should be one recognized by the cell chosen for expression of the chimeric polypeptide. It can be prokaryotic, eukaryotic or a combination of the two. In general, the inclusion of a signal sequence depends on whether it is desired to secrete the chimeric polypeptide as disclosed herein from the recombinant cells in which it is made. If the chosen cells are prokaryotic, it generally is preferred that the DNA sequence not encode a signal sequence. If the chosen cells are eukaryotic, it generally is preferred that a signal sequence be included.

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric polypeptide) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

Exemplary isolated nucleic acid molecules of the present disclosure can include fragments not found as such in the natural state. Thus, this disclosure encompasses recombinant molecules, such as those in which a nucleic acid sequence (for example, a sequence encoding a chimeric polypeptide disclosed herein) is incorporated into a vector (e.g., a plasmid or viral vector) or into the genome of a heterologous cell (or the genome of a homologous cell, at a position other than the natural chromosomal location).

Provided herein are methods of incorporation of one or more nucleic acid encoding one or more chimeric polypeptide of the present disclosure into the genome of a heterologous cell. Such methods preferably involve Homology-Directed Recombination (HDR), which requires the introduction of a double strand break (DSB) in the target genomic location using specifically designed endonucleases or nickases. The “donor” nucleic acid comprising the one or more chimeric polypeptide is then introduced in the genome of the cell by HDR.

As used herein, “donor” nucleic acid are used interchangeably and refers to a nucleic acid that corresponds to a fragment of the endogenous targeted gene of a cell (in some embodiments the entire targeted gene), but which includes the one or more chimeric polypeptides and other sequences necessary for the expression of said chimeric polypeptides (such as, but not limited to, a promoter and/or an enhancer). The donor nucleic acid must be of sufficient size and similarity to permit homologous recombination with the targeted gene. The donor nucleic acid may be provided for example as a single-stranded oligodeoxynucleotides (ssODN), as a PCR product (amplicon) or within a vector. Preferably, the donor nucleic acid will include modifications with respect to the endogenous gene which i) precludes it from being cut by a gRNA once integrated in the genome of a cell and/or which facilitate the detection of the introduction of the donor nucleic acid by homologous recombination.

The CRISPR/Cas system is an efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/CAS system can thereby be engineered to cleave virtually any DNA sequence by redesigning the gRNA in cell lines (such as 293T cells), primary cells, and T cells. The CRISPR/CAS system can simultaneously target multiple genomic loci by co-expressing a single CAS9 protein with two or more gRNAs, making this system suited for HDR when provided with a donor sequence.

The present disclosure provides methods for generating cells expressing one or more chimeric polypeptide by introducing a Cas expression vector and a guide nucleic acid sequence specific for a gene into a cell. In another embodiment, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, Cpf1 (or Cas12a), other nucleases known in the art, and any combination thereof.

In one embodiment, inducing the Cas expression vector comprises exposing the cell to an agent that activates an inducible promoter in the Cas expression vector. In such an embodiment, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

The guide nucleic acid sequence is specific for a gene and targets that gene for Cas endonuclease-induced double strand breaks. The sequence of the guide nucleic acid sequence may be within a loci of the gene. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length. The guide nucleic acid sequence may be specific for any genomic locus in the cell.

The guide nucleic acid sequence includes a RNA sequence, a DNA sequence, a combination thereof (a RNA-DNA combination sequence), or a sequence with synthetic nucleotides. The guide nucleic acid sequence can be a single molecule or a double molecule. In one embodiment, the guide nucleic acid sequence comprises a single guide RNA.

Provided herein are methods of incorporation of one or more nucleic acid encoding one or more chimeric polypeptide of the present disclosure into the genome of a heterologous cell by HDR, using other various types of endonucleases or nickases. Non-limiting examples of endonucleases and nickases include meganucleases, Zinc Finger Nucleases (ZFNs), Transcription Activator-Like Effector nucleases (TALENs) (Gaj T, Gersbach C A, & Barbas C F, 3rd (2013) ZFN, TALEN, and CRISPR/Cas-based methods for genome engineering. Trends Biotechnol 31 (7): 397-405; Arnould S, et al. (2011) The 1-CreI meganuclease and its engineered derivatives: applications from cell modification to gene therapy. Protein engineering, design & selection: PEDS 24 (1-2): 27-31). These endonucleases and nickases can be used to introduce a nucleic acid encoding for one or more chimeric polypeptide, within a targeted genomic sequence by HDR, in accordance with the present invention, when provided with a donor sequence comprising an expression cassette for said one or more chimeric polypeptides.

### Host Cells

One aspect of the disclosure is a cell that contains a chimeric polypeptide, and/or contain a nucleic acid that encodes any chimeric polypeptide disclosed herein. An embodiment is a recombinant cell including the chimeric polypeptides and STMD receptors as disclosed herein, and its progeny. In some embodiments, the recombinant cells include a recombinant nucleic acid as disclosed herein.

Cell cultures containing at least one recombinant cell as disclosed herein are also within the scope of the present disclosure. It should be understood that not all progeny are exactly identical to the parental cell (due to deliberate or inadvertent mutations or differences in environment); however, such altered progeny are included in these terms, so long as the progeny retain the same functionality as that of the originally transformed cell.

The nucleic acid of the present disclosure can be introduced into a host cell, such as, for example, a human T lymphocyte, to produce a recombinant cell containing the nucleic acid molecule. Accordingly, some embodiments of the disclosure relate to methods for making a recombinant cell, including (a) providing a cell capable of protein expression and (b) contacting the provided cell with a recombinant nucleic acid of the disclosure.

Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell (Tx), a cytotoxic T cell (Tcm), or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+T cytotoxic lymphocyte cell selected from the group consisting of naive CD8+ T cells, central memory CD8+ T cells, effector memory CD8+ T cells, and bulk CD8+ T cells. In some embodiments of the cell, the cell is a CD4+T helper lymphocyte cell selected from the group consisting of naive CD4+ T cells, central memory CD4+ T cells, effector memory CD4+ T cells, and bulk CD4+ T cells. In some embodiments, the cell can be obtained by leukapheresis performed on a sample obtained from a subject. In some embodiments, the subject is a human patient.

In some embodiments, the recombinant cell further includes a first and a second nucleic acid molecule as disclosed herein, wherein the first nucleic acid molecule and the second nucleic acid molecule do not have the same sequence. In some embodiments, the recombinant cell further includes a first and a second chimeric polypeptide or STMD receptor as disclosed herein, wherein the first chimeric polypeptide or STMD receptor and the second chimeric polypeptide or STMD receptor do not have the same sequence. In some embodiments, the first chimeric polypeptide or STMD receptor modulates the expression and/or activity of the second chimeric polypeptide or STMD receptor.

In some embodiments, the recombinant cell further includes an expression cassette encoding a protein of interest operably linked to a promoter, wherein expression of the protein of interest is modulated by the chimeric receptor transcriptional regulator. In some embodiments, the protein of interest is heterologous to the recombinant cell. A heterologous protein is one that is not normally found in the cell, e.g., not normally produced by the cell. In principle, there are no particular limitations with regard to suitable proteins whose expression can be modulated by the chimeric receptor transcriptional regulator. Exemplary types of proteins suitable for use with the compositions and methods disclosed herein include cytokines, cytotoxins, chemokines, immunomodulators, pro-apoptotic factors, anti-apoptotic factors, hormones, differentiation factors, dedifferentiation factors, immune cell receptors, or reporters. In some embodiments, the immune cell receptor is a T-cell receptor (TCR).

In some embodiments, the immune cell receptor is a chimeric antigen receptor (CAR). In some embodiments, the expression cassette encoding the protein of interest is incorporated into the same nucleic acid molecule that encodes the chimeric receptor of the disclosure. In some embodiments, the expression cassette encoding the protein of interest is incorporated into a second expression vector that is separate from the nucleic acid molecule encoding the chimeric receptor of the disclosure. In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the chimeric polypeptides, STMD receptors, nucleic acids, and recombinant cells of the disclosure can be incorporated into compositions, including pharmaceutical compositions. Such compositions typically include the chimeric polypeptides, STMD receptors, nucleic acids, and/or recombinant cells, and a pharmaceutically acceptable excipient, e.g., carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to allow easy syringability. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound (e.g., chimeric polypeptides, STMD receptors, nucleic acids, and/or recombinant cells of the disclosure) can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches, and the like, can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In the event of administration by inhalation, the subject chimeric polypeptides and STMD receptors of the disclosure are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of the subject chimeric polypeptides and STMD receptors of the disclosure can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the chimeric polypeptides and STMD receptors of the disclosure can also be prepared in the form of suppositories (e.g., with conventional suppository bases such as cocoa butter and other glycerides) or retention enemas for rectal delivery.

In some embodiments, the chimeric polypeptides and STMD receptors of the disclosure can also be administered by transfection or infection using methods known in the art, including but not limited to the methods described in McCaffrey et al. (Nature 418:6893, 2002), Xia et al. (Nature Biotechnol. 20:1006-1010, 2002), or Putnam (Am. J. Health Syst. Pharm. 53:151-160, 1996, erratum at Am. J. Health Syst. Pharm. 53:325, 1996).

In some embodiments, the subject chimeric polypeptides and STMD receptors of the disclosure are prepared with carriers that will protect the recombinant polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. As described in greater detail below, the chimeric polypeptides and STMD receptors of the present disclosure may also be modified to achieve extended duration of action such as by PEGylation, acylation, Fc fusions, linkage to molecules such as albumin, etc. In some embodiments, the recombinant polypeptides can be further modified to prolong their half-life in vivo and/or ex vivo. Non-limiting examples of known strategies and methodologies suitable for modifying the recombinant polypeptides of the disclosure include (1) chemical modification of a recombinant polypeptide described herein with highly soluble macromolecules such as polyethylene glycol (“PEG”) which prevents the recombinant polypeptides from contacting with proteases; and (2) covalently linking or conjugating a recombinant polypeptide described herein with a stable protein such as, for example, albumin. Accordingly, in some embodiments, the chimeric polypeptides and STMD receptors of the disclosure can be fused to a stable protein, such as, albumin. For example, human albumin is known as one of the most effective proteins for enhancing the stability of polypeptides fused thereto and there are many such fusion proteins reported.

In some embodiments, the pharmaceutical compositions of the disclosure include one or more pegylation reagents. As used herein, the term “PEGylation” refers to modifying a protein by covalently attaching polyethylene glycol (PEG) to the protein, with “PEGylated” referring to a protein having a PEG attached. A range of PEG, or PEG derivative sizes with optional ranges of from about 10,000 Daltons to about 40,000 Daltons may be attached to the recombinant polypeptides of the disclosure using a variety of chemistries. In some embodiments, the average molecular weight of said PEG, or PEG derivative, is about 1 kD to about 200 kD such as, e.g., about 10 kD to about 150 kD, about 50 kD to about 100 kD, about 5 kD to about 100 kD, about 20 kD to about 80 kD, about 30 kD to about 70 kD, about 40 kD to about 60 kD, about 50 kD to about 100 kD, about 100 kD to about 200 kD, or about 1 150 kD to about 200 kD. In some embodiments, the average molecular weight of said PEG, or PEG derivative, is about 5 kD, about 10 kD, about 20 kD, about 30 kD, about 40 kD, about 50 kD, about 60 kD, about 70 kD, or about 80 kD. In some embodiments, the average molecular weight of said PEG, or PEG derivative, is about 40 kD. In some embodiments, the pegylation reagent is selected from methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-Succinimidyl Glutarate (mPEG-SG), mPEG-N-hydroxyl-succinimide (mPEG-NHS), mPEG-tresylate and mPEG-aldehyde. In some embodiments, the pegylation reagent is polyethylene glycol; for example said pegylation reagent is polyethylene glycol with an average molecular weight of 20,000 Daltons covalently bound to the N-terminal methionine residue of the recombinant polypeptides of the disclosure. In some embodiments, the pegylation reagent is polyethylene glycol with an average molecular weight of about 5 kD, about 10 kD, about 20 kD, about 30 kD, about 40 kD, about 50 kD, about 60 kD, about 70 kD, or about 80 kD covalently bound to the N-terminal methionine residue of the chimeric polypeptides and STMD receptors of the disclosure. In some embodiments, the pegylation reagent is polyethylene glycol with an average molecular weight of about 40 kD covalently bound to the N-terminal methionine residue of the chimeric polypeptides and STMD receptors of the disclosure.

Accordingly, in some embodiments, the chimeric polypeptides and STMD receptors of the disclosure are chemically modified with one or more polyethylene glycol moieties, e.g., PEGylated; or with similar modifications, e.g. PASylated. In some embodiments, the PEG molecule or PAS molecule is conjugated to one or more amino acid side chains of the disclosed recombinant polypeptide. In some embodiments, the PEGylated or PASylated polypeptide contains a PEG or PAS moiety on only one amino acid. In other embodiments, the PEGylated or PASylated polypeptide contains a PEG or PAS moiety on two or more amino acids, e.g., attached to two or more, five or more, ten or more, fifteen or more, or twenty or more different amino acid residues. In some embodiments, the PEG or PAS chain is 2000, greater than 2000, 5000, greater than 5,000, 10,000, greater than 10,000, greater than 10,000, 20,000, greater than 20,000, and 30,000 Da. The PASylated polypeptide may be coupled directly to PEG or PAS (e.g., without a linking group) through an amino group, a sulfhydryl group, a hydroxyl group, or a carboxyl group. In some embodiments, the recombinant polypeptide of the disclosure is covalently bound to a polyethylene glycol with an average molecular weight of 20,000 Daltons. In some embodiments, the recombinant polypeptide of the disclosure is covalently bound to a polyethylene glycol with an average molecular weight ranging from about 1 kD to about 200 kD such as, e.g., about 10 kD to about 150 kD, about 50 kD to about 100 kD, about 5 kD to about 100 kD, about 20 kD to about 80 kD, about 30 kD to about 70 kD, about 40 kD to about 60 kD, about 50 kD to about 100 kD, about 100 kD to about 200 kD, or about 1 150 kD to about 200 kD. In some embodiments, the recombinant polypeptide of the disclosure is covalently bound to a polyethylene glycol with an average molecular weight of about 5 kD, about 10 kD, about 20 kD, about 30 kD, about 40 kD, about 50 kD, about 60 kD, about 70 kD, or about 80 kD. In some embodiments, the recombinant polypeptide of the disclosure is covalently bound to a polyethylene glycol with an average molecular weight of about 40 kD.

### Methods of the Disclosure

### Method for the Treatment of a Health Condition in an Individual in Need Thereof

Provided herein are methods for the treatment of a health condition in an individual in need thereof. Administration of any one of the therapeutic compositions described herein, e.g., chimeric polypeptides, STMD receptors, nucleic acids, recombinant cells, and pharmaceutical compositions, can be used to treat patients in the treatment of relevant diseases, such as cancers and chronic infections. In some embodiments, the chimeric polypeptides, STMD receptors, nucleic acids, recombinant cells, and pharmaceutical compositions as described herein can be incorporated into therapeutic agents for use in methods of treating an individual who has, who is suspected of having, or who may be at high risk for developing one or more autoimmune disorders or health diseases associated with checkpoint inhibition. Exemplary autoimmune disorders and health diseases can include, without limitation, cancers and chronic infection.

In some embodiments, the methods include administering to the individual an effective number of the recombinant cells disclosed herein, wherein the recombinant cells inhibit an activity of the target cells in the individual. Generally, the target cells of the disclosed methods can be any cell type in an individual and can be, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a multiple myeloma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell.

In some embodiments, the methods of the disclosure involve administering an effective amount of the recombinants cells of the disclosure to an individual in need of such treatment. This administering step can be accomplished using any method of implantation delivery in the art. For example, the recombinant cells of the disclosure can be infused directly in the individual's bloodstream or otherwise administered to the individual.

In some embodiments, the methods disclosed herein include administering, which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even the lifetime of the individual, i.e., long-term engraftment.

When provided prophylactically, the recombinant cells described herein can be administered to an individual in advance of any symptom of a disease or condition to be treated. Accordingly, in some embodiments the prophylactic administration of a recombinant cell population prevents the occurrence of symptoms of the disease or condition.

When provided therapeutically in some embodiments, recombinant cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5.times.10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source. In some embodiments, the recombinant cells are expanded in culture prior to administration to an individual in need thereof.

In some embodiments, the delivery of a recombinant cell composition (e.g., a composition including a plurality of recombinant cells according to any of the cells described herein) into an individual by a method or route results in at least partial localization of the cell composition at a desired site. A composition including recombinant cells can be administered by any appropriate route that results in effective treatment in the individual, e.g., administration results in delivery to a desired location in the individual where at least a portion of the composition delivered, e.g., at least 1.×.10⁴ cells, is delivered to the desired site for a period of time. Modes of administration include injection, infusion, instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a preferred mode of administration.

In some embodiments, the recombinant cells are administered systemically, e.g., via infusion or injection. For example, a population of recombinant cells are administered other than directly into a target site, tissue, or organ, such that it enters, the individual's circulatory system and, thus, is subject to metabolism and other similar biological processes.

The efficacy of a treatment including any of the compositions provided herein for the treatment of a disease or condition can be determined by a skilled clinician. However, one skilled in the art will appreciate that a treatment is considered effective if any one or all of the signs or symptoms or markers of disease are improved or ameliorated. Efficacy can also be measured by failure of an individual to worsen as assessed by decreased hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular beneficial effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments of the disclosed methods, the individual is a mammal. In some embodiments, the mammal is a human. In some embodiments, the individual has or is suspected of having a disease associated with inhibition of cell signaling mediated by a cell surface ligand or antigen. The diseases suitable for being treated by the compositions and methods of the disclosure include, but are not limited to, cancers, autoimmune diseases, inflammatory diseases, and infectious diseases. In some embodiments, the disease is a cancer or a chronic infection.

### Additional Therapies

As discussed above, the recombinant cells, and pharmaceutical compositions described herein can be administered in combination with one or more additional therapeutic agents such as, for example, chemotherapeutics or anti-cancer agents or anti-cancer therapies. Administration “in combination with” one or more additional therapeutic agents includes simultaneous (concurrent) and consecutive administration in any order. In some embodiments, the one or more additional therapeutic agents, chemotherapeutics, anti-cancer agents, or anti-cancer therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

In some embodiments, the first therapy and the additional therapy (e.g., second therapy) are administered concomitantly. In some embodiments, the first therapy and the additional therapy are administered separately or sequentially. The therapies can be administered in the same or in different compositions.

### Method for Modulating an Activity of a Cell

In one aspect, some embodiments of the disclosure relate to methods for modulating an activity of a target cell in an individual, the methods include providing a recombinant cell of the disclosure and contacting the recombinant cell with a selected ligand wherein binding of the selected ligand to the extracellular binding domain of the chimeric polypeptide of the STMD receptors of the disclosure, induces cleavage of a ligand-inducible proteolytic cleavage site and releases the transcriptional regulator whereby the released transcriptional regulator modulates an activity of the recombinant cell. In some embodiments, the released transcriptional regulator modulates differentiation of the cell, and wherein the cell is an immune cell, a stem cell, a progenitor cell, or a precursor cell.

In some embodiments the released transcriptional regulator modulates expression of a gene product of the cell. In some embodiments, the released transcriptional regulator modulates expression of an endogenous gene product. In some embodiments, the released transcriptional regulator modulates expression of a heterologous gene product. In some embodiments, the gene product of the cell is a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, an RNA guided nuclease, a site-specific nuclease, a T cell receptor, a toxin, a toxin derived protein, a transcriptional activator, a transcriptional repressor, a translation regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

Some embodiments of the disclosure relate to methods for inhibiting an activity of a target cell in an individual, the methods include administering to the individual a first therapy including one or more of nucleic acids, recombinant cells, and pharmaceutical compositions as disclosed herein, wherein the first therapy inhibits the target cell. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cells disclosed herein, wherein the recombinant cells inhibit an activity of the target cells in the individual. Generally, the target cells of the disclosed methods can be any cell type in an individual and can be, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a multiple melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, a hematological malignancy cell, a solid tumor cell or a sarcoma cell. In some embodiments, the target cell is a pathogenic cell.

The target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, contacting the cell is carried out in vivo, ex vivo or in vitro.

For example, the activity of the cell can be: expression of a selected gene of the cell, proliferation of the cell, apoptosis of the cell, non-apoptotic death of the cell, differentiation of the cell, dedifferentiation of the cell, migration of the cell, secretion of a molecule from the cell, cellular adhesion of the cell, and cytolytic activity of the cell.

### Method of Inducing T Cell Signaling and Gene Regulation in a T Cell

The disclosure also provides a method for inducing T cell signaling and gene regulation in a T cell including providing a vector comprising the chimeric polypeptide of the disclosure and transducing a T cell with the vector wherein binding of a selected ligand to the extracellular ligand-binding domain of the chimeric polypeptide induces intracellular signaling and release of the transcriptional regulator.

### Methods of Making the Recombinant Cells of the Disclosure

The disclosure also provides a method for making the recombinant cells of the disclosure. In some embodiments, the methods for making the recombinant cells include providing a cell capable of protein expression; and contacting the provided cell with a recombinant nucleic acid molecules of the disclosure. Contacting can include transducing the cells with the recombinant nucleic acid molecules by any method known to a skilled in the art. Some of said methods are described supra.

The current disclosure also provides the use of the compositions of the disclosure for the treatment of a disease. In some embodiments, the use is of the chimeric polypeptides of the disclosure. In some embodiments, the use is of the recombinant nucleic acid molecules of the disclosure. In some embodiments, the use is of the recombinant cell of the disclosure. In some embodiments, the use is of the STMDs of the disclosure.

The current disclosure also provides the use of any of the compositions, methods, kits and systems herein in the or for the manufacture of a medicament for the treatment of a disease. In some embodiments, the disease is cancer. In some embodiments, the cancer is a solid tumor or a hematological malignancy. In some embodiments, the hematological malignancy is multiple myeloma.

### Systems and Kits

Systems or kits of the present disclosure include one or more of any of the chimeric polypeptides, STMD receptors, recombinant nucleic acids, recombinant cells, or pharmaceutical compositions disclosed herein as well as syringes (including pre-filled syringes) and/or catheters (including pre-filled syringes) used to administer any of the chimeric polypeptides, STMD receptors, recombinant nucleic acids, recombinant cells, or pharmaceutical compositions to an individual. The kits also include written instructions for using of any of the chimeric polypeptides, STMD receptors, recombinant nucleic acids, recombinant cells, or pharmaceutical compositions disclosed herein as well as syringes and/or catheters for use with their administration.

Accordingly provided herein is a system for modulating an activity of a cell, killing a target cancer cell, or treating a disease in an individual in need thereof, wherein the system includes one or more of the following: a) a chimeric polypeptide according to any one of claims 1 to 34; b) a recombinant nucleic acid molecule according to any one of claims 40 to 41; c) a recombinant cell according to any one of claims 46 to 52; and/or d) a pharmaceutical composition of the disclosure.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, N L: Elsevier Sciences B. V., the disclosures of which are incorporated herein by reference.

Additional embodiments are disclosed in further detail in the following examples, which are provided by way of illustration and are not in any way intended to limit the scope of this disclosure or the claims.

### Example 1

### Design and Construction of Chimeric Polypeptide and Response Element Constructs

This Example describes the design and construction of receptors with STMDs.

The chimeric polypeptides described herein were built by fusing the CD19 scFv (Porter et al. 2011) to the corresponding receptor scaffold and Gal4 DBD VP64. All receptors contained an N-terminal CD8α signal peptide (MALPVTALLLPLALLLHAARP) (SEQ ID NO: 31) for membrane targeting and a myc-tag (EQKLISEEDL) (SEQ ID NO: 32) for suitable determination of surface expression with an antibody conjugated to a fluorescent dye (α-myc A647®, Cell Signaling Technology, Cat #2233).

The transcriptional regulator GAL4-VP64 used in these experiments contained a DNA domain from yeast GAL4 transcription factor fused to an activation domain VP64, which consists of a tetrameric repeat of the minimal activation domain (amino acids 437-447) of the herpes simplex protein VP16. The receptors were cloned into a modified pHR′ SIN:CSW vector containing a PGK promoter for all primary T cell experiments (SEQ ID NO: 1).

The pHR′ SIN:CSW vector was also modified to make the response element plasmids. Five copies of the Gal4 DNA binding domain target sequence (GGAGCACTGTCCTCCGAACG) (SEQ ID NO: 33) were cloned 5′ to a minimal pybTATA promoter. Also included in the response element plasmids is a PGK promoter that constitutively drives mCitrine expression to easily identify transduced T cells. For all inducible BFP vectors, BFP was cloned via a BamHI site in the multiple cloning site 3′ to the Gal4 response elements. For all inducible CAR vectors, the CARs were tagged c-terminally with GFP and were cloned via a BamHI site in the multiple cloning site 3′ to the Gal4 response elements. All constructs were cloned via Infusion cloning (Clontech #ST0345).

BamHI homology sites were added to the following receptor sequences (provided as nucleotide sequences) and inserted into the above lentiviral transduction vector. Description of individual components follow the contiguous sequence. The following chimeric polypeptides listed in Table 1 were constructed.

TABLE 1

| SEQ ID | | Hinge | | | |
|---|---|---|---|---|---|
| NO | ECD | Domain | STMD | JMD | TR |
| 34 | Anti- | CD8α | GG0 | Notch2 | Gal4VP64 |
| | CD19scFv- | Hinge | | JMD | |
| 35 | Anti- | CD8α | GG1 | Notch2 | Gal4VP64 |
| | CD19scFv- | Hinge | | JMD | |
| 36 | Anti- | CD8α | GG2 | Notch2 | Gal4VP64 |
| | CD19scFv- | Hinge | | JMD | |
| 37 | Anti- | CD8α | GG3 | Notch2 | Gal4VP64 |
| | CD19scFv- | Hinge | | JMD | |
| 38 | Anti- | CD8α | GG4 | Notch2 | Gal4VP64 |
| | CD19scFv- | Hinge | | JMD | |
| 39 | Anti- | CD8α | GG5 | Notch2 | Gal4VP64 |
| | CD19scFv- | Hinge | | JMD | |
| 40 | Anti- | CD8α | GG6 | Notch2 | Gal4VP64 |
| | CD19scFv- | Hinge | | JMD | |
| 41 | Anti- | CD8α | GG7 | Notch2 | Gal4VP64 |
| | CD19scFv- | Hinge | | JMD | |
| 42 | Anti- | CD8α | GG8 | Notch2 | Gal4VP64 |
| | CD19scFv- | Hinge | | JMD | |
| 43 | Anti- | CD8α | GG9 | Notch2 | Gal4VP64 |
| | CD19scFv- | Hinge | | JMD | |
| 44 | Anti- | CD8α | GG10 | Notch2 | Gal4VP64 |
| | CD19scFv- | Hinge | | JMD | |
| 45 | Anti- | CD8α | GG11 | Notch2 | Gal4VP64 |
| | CD19scFv- | Hinge | | JMD | |
| 46 | Anti- | CD8α | GG12 | Notch2 | Gal4VP64 |
| | CD19scFv- | Hinge | | JMD | |
| 47 | Anti- | CD8α | GG13 | Notch2 | Gal4VP64 |
| | CD19scFv- | Hinge | | JMD | |
| 78 | Anti- | CD8α | GG14 | Notch2 | Gal4VP64 |
| | CD19scFv- | Hinge | | JMD | |
| 50 | Anti- | CD8α | GG15 | Notch2 | Gal4VP64 |
| | CD19scFv- | Hinge | | JMD | |
| 51 | Anti- | CD8α | GG16 | Notch2 | Gal4VP64 |
| | CD19scFv- | Hinge | | JMD | |
| 52 | Anti- | CD8α | GG17 | Notch2 | Gal4VP64 |
| | CD19scFv- | Hinge | | JMD | |
| 53 | Anti- | CD8α | GG18 | Notch2 | Gal4VP64 |
| | CD19scFv- | Hinge | | JMD | |
| 54 | Anti- | CD8α | GG19 | Notch2 | Gal4VP64 |
| | CD19scFv- | Hinge | | JMD | |
| 55 | Anti- | CD8α | GG20 | Notch2 | Gal4VP64 |
| | CD19scFv- | Hinge | | JMD | |

### Example 2

### Primary Human T-Cell Isolation and Culture

This Example describes the isolation and culture of primary human T cells that were subsequently used in various cell transduction experiments described in Example 3 below.

Primary CD4+ and CD8+ T cells were isolated from anonymous donor blood after apheresis by negative selection (STEMCELL Technologies #15062 & 15063). Blood was obtained from Blood Centers of the Pacific (San Francisco, CA) as approved by the University Institutional Review Board. T cells were cryopreserved in RPMI-1640 (UCSF cell culture core) with 20% human AB serum (Valley Biomedical Inc., #HP1022) and 10% DMSO. After thawing, T cells were cultured in human T cell medium consisting of X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

### Example 3

### Lentiviral Transduction of Human T Cells

The Example describes a general protocol used for lentiviral transduction of human T cells with pantropic VSV-G pseudotyped lentivirus.

Pantropic VSV-G pseudotyped lentivirus was produced via transfection of Lenti-X 293T cells (Clontech #11131D) with a pHR′ SIN:CSW transgene expression vector and the viral packaging plasmids pCMVdR8.91 and pMD2.G using Mirus TransIT-Lenti (Mirus #MIR 6606). Primary T cells were thawed the same day, and after 24 hours in culture, were stimulated with Human T-Activator CD3/CD28 Dynabeads (Life Technologies #11131D) at a 1:3 cell:bead ratio. At 48 hours, viral supernatant was harvested and the primary T cells were exposed to the virus for 24 hours. At day 5 post T cell stimulation, the Dynabeads were removed, and the T cells were sorted with a Beckton Dickinson (BD) FACs ARIA II and expanded for use in assays.

### Example 4

### Cancer Cell Lines

This Example describes the generation of myelogenous leukemia cells expressing CD19 at equivalent levels as Daudi tumors.

The cancer cell lines used were K562 myelogenous leukemia cells (ATCC #CCL-243). K562s were lentivirally transduced to stably express human CD19 at equivalent levels as Daudi tumors. CD19 levels were determined by staining the cells with α-CD19 APC (Biolegend #302212). All cell lines were sorted for expression of the transgenes.

### Example 5

### In Vitro Stimulation of Primary T Cells

This Example describes the stimulation of primary T cells in vitro.

For all in vitro T cell stimulations, 1×10⁵ T cells were co-cultured with target cells at a 1:1 ratio in U-bottom 96-well tissue culture plates. The cultures were analyzed at 24 hours or as indicated for reporter activation and/or target cell killing with a BD Fortessa X-50. All flow cytometry analysis was performed in FlowJo software (TreeStar).

### Example 6

### Testing of the Synthetic Polyvaline TMD-Based Receptors

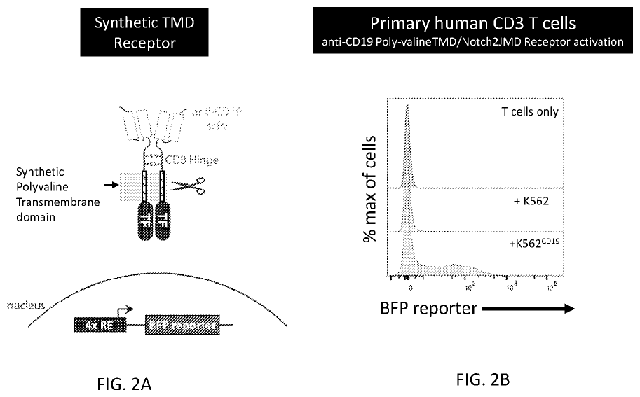

Primary human CD3+ T-cells were activated with anti-CD3/anti-CD28 Dynabeads (Gibco) and transduced with a lentiviral construct expressing a multi-chain receptor construct, and another lentiviral construct containing the transcriptional reporter construct. Receptor expression was measured using an AlexaFluor647-tagged anti-myc antibody (Cell Signaling) against the myc tag on the binder (CD19scFv)-containing chain. Reporter expression was measured through a constitutive mCitrine gene found on the reporter plasmid. Double positive cells were sorted for on Day 5 post initial T-cell stimulation and expanded further for activation testing. For measuring receptor activity, 1E5 double positive T-cells expressing anti-CD19 receptors are co-cultured with: no additions (red), 1E5 K562 cells (blue), or 1E5 CD19+K562 cells (yellow) for 48 hours. Transcriptional activation of an inducible BFP reporter gene was subsequently measured using a Fortessa X-50 (BD) (FIG. 2B)

### Example 7

### Testing of the Synthetic Polyvaline TMD-Based Receptors with a Destabilizing Residue

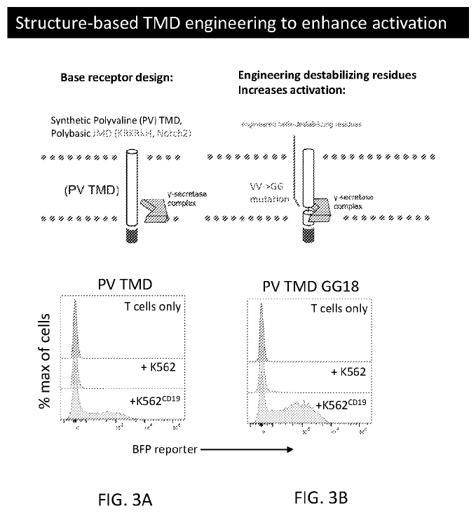

Primary human CD3+ T-cells were activated with anti-CD3/anti-CD28 Dynabeads (Gibco) and transduced with a lentiviral construct expressing a multi-chain receptor construct, and another lentiviral construct containing the transcriptional reporter construct. Receptor expression was measured using an AlexaFluor647-tagged anti-myc antibody (Cell Signaling) against the myc tag on the binder (CD19scFv)-containing chain. Reporter expression was measured through a constitutive mCitrine gene found on the reporter plasmid. Double positive cells were sorted for on Day 5 post initial T-cell stimulation and expanded further for activation testing. For measuring receptor activity, 1E5 double positive T-cells expressing anti-CD19 receptors were co-cultured with: no additions (red), 1E5 K562 cells (blue), or 1E5 CD19+K562 cells (yellow) for 48 hours. Transcriptional activation of an inducible BFP reporter gene was subsequently measured using a Fortessa X-50 (BD). Here, destabilizing residues at position 18-19 of the polyvaline TMD (GG18, FIG. 3B) increases transcriptional activation compared to without destabilizing residues (FIG. 3A).

### Example 8

### Testing Destabilizing Residues within the First N-Terminal Residues of the of the Synthetic Polyvaline TMD-Based Receptors

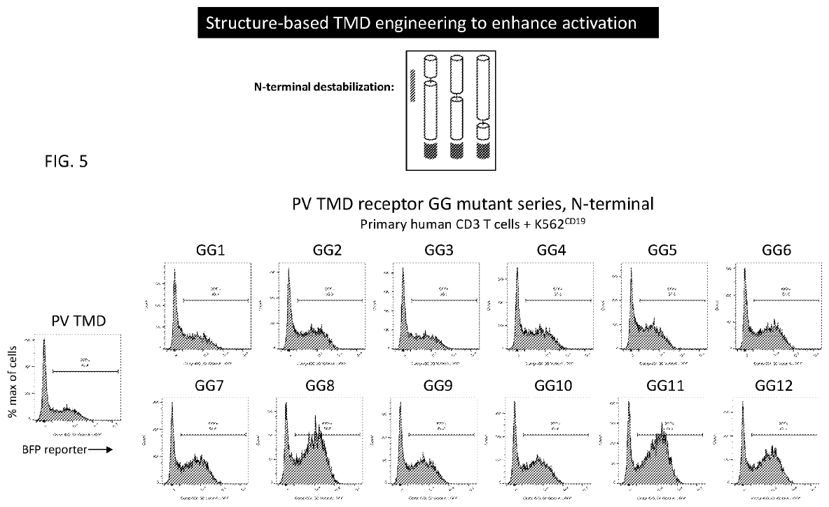

Primary human CD3+ T-cells were activated with anti-CD3/anti-CD28 Dynabeads (Gibco) and transduced with a lentiviral construct expressing a multi-chain receptor construct, and another lentiviral construct containing the transcriptional reporter construct. Receptor expression was measured using an AlexaFluor647-tagged anti-myc antibody (Cell Signaling) against the myc tag on the binder (CD19scFv)-containing chain. Reporter expression was measured through a constitutive mCitrine gene found on the reporter plasmid. Double positive cells were sorted for on Day 5 post initial T-cell stimulation and expanded further for activation testing. For measuring receptor activity, 1E5 double positive T-cells expressing anti-CD19 receptors were co-cultured with 1E5 CD19+K562 cells (yellow) for 48 hours. Transcriptional activation of an inducible BFP reporter gene was subsequently measured using a Fortessa X-50 (BD). Here, destabilizing residues at positions 1-2 (GG1) through 12-13 (GG12) showed about equivalent or higher activity compared to the original polyvaline TMD (PV TMD) (FIG. 5).

### Example 9

### Testing Destabilizing Residues within the Last C-Terminal Residues of the of the Synthetic Polyvaline TMD-Based Receptors

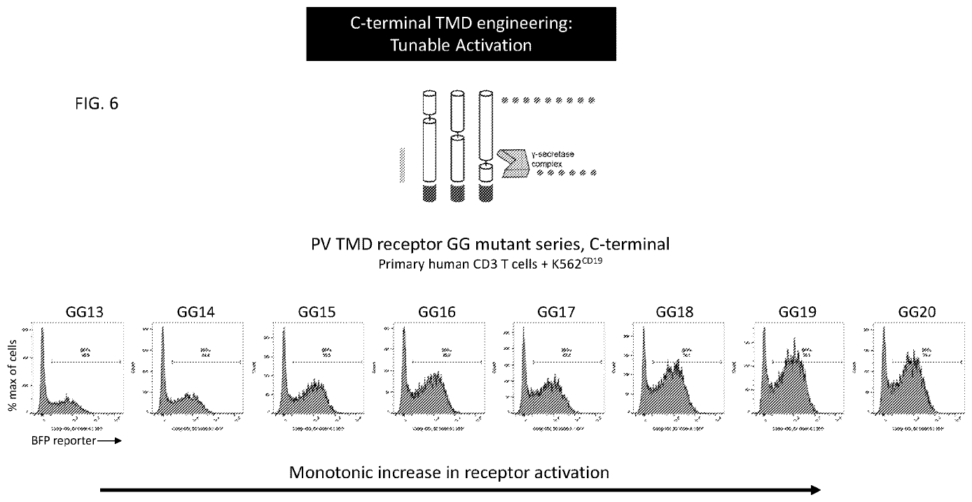

Primary human CD3+ T-cells were activated with anti-CD3/anti-CD28 Dynabeads (Gibco) and transduced with a lentiviral construct expressing a multi-chain receptor construct, and another lentiviral construct containing the transcriptional reporter construct. Receptor expression was measured using an AlexaFluor647-tagged anti-myc antibody (Cell Signaling) against the myc tag on the binder (CD19scFv)-containing chain. Reporter expression is measured through a constitutive mCitrine gene found on the reporter plasmid. Double positive cells are sorted for on Day 5 post initial T-cell stimulation and expanded further for activation testing. For measuring receptor activity, 1E5 double positive T-cells expressing anti-CD19 receptors were co-cultured with 1E5 CD19+K562 cells (yellow) for 48 hours. Transcriptional activation of an inducible BFP reporter gene was subsequently measured using a Fortessa X-50 (BD). Here, destabilizing residues at positions 13-14 (GG13) through 20-21 (GG20) showed a monotonic increase in receptor activation, showing more activity as the GG mutants are more C-terminal. (FIG. 6).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

INFORMAL SEQUENCE LISTING

| SEQ | | Description* |
|---|---|---|
| ID NO | Sequence | |
| 1 | gggtaggggaggcgcttttcccaaggcagtctggagcatgcgctttagcagccccgctgggcacttggcgctacac | Modificd |
| | aagtggcctctggcctcgcacacattccacatccaccggtaggcgccaaccggctccgttctttggtggccccttcgc | pHR'SIN:CSW vector |
| | gccaccttctactcctcccctagtcaggaagttcccccccgccccgcagctcgcgtcgtgcaggacgtgacaaatgg | used for lentiviral |
| | aagtagcacgtctcactagtctcgtgcagatggacagcaccgctgagcaatggaagcgggtaggcctttggggcag | transduction and |
| | cggccaatagcagctttgctccttcgctttctgggctcagaggctgggaaggggtgggtccgggggcgggctcagg | receptor expression. |
| | ggcgggctcaggggcggggcgggcgcccgaaggtcctccggaggcccggcattctgcacgcttcaaaagcgca | Receptors were |
| | cgtctgccgcgctgttctcctcttcctcatctccgggcctttcgaattctcacgcgtcaagtggagcaaggcaggtgga | inserted by In-fusion |
| | cagtgGGATCCttgacttgcggccgcaactcccacctgcaacatgcgtgactgactgaggccgcgactctaga | cloning (Clontech) into |
| | gtcgacctgcaggcatgcaagcttgatatcaagcttatcgataatcaacctctggattacaaaatttgtgaaagattgac | the BamHI (GGATCC) |
| | tggtattcttaactatgttgctccttttacgctatgtggatacgctgctttaatgcctttgtatcatgctattgcttcccgtatgg | site downstream a PGK |
| | ctttcattttctcctccttgtataaatcctggttgctgtctctttatgaggagttgtggcccgttgtcaggcaacgtggcgtg | promoter sequence |
| | gtgtgcactgtgtttgctgacgcaacccccactggttggggcattgccaccacctgtcagctcctttccgggactttcg | |
| | ctttccccctccctattgccacggcggaactcatcgccgcctgccttgcccgctgctggacaggggctcggctgttgg | |
| | gcactgacaattccgtggtgttgtcggggaaatcatcgtcctttccttggctgctcgcctgtgttgccacctggattctgc | |
| | gcgggacgtccttctgctacgtcccttcggccctcaatccagcggaccttccttcccgcggcctgctgccggctctgc | |
| | ggcctcttccgcgtcttcgccttcgccctcagacgagtcggatctccctttgggccgcctccccgcatcgataccgtcg | |
| | accaaggcagctgtagatcttagccactttttaaaagaaaaggggggactggaagggctaattcactcccaacgaag | |
| | acaagatctgctttttgcttgtactgggtctctctggttagaccagatctgagcctgggagctctctggctaactaggga | |
| | acccactgcttaagcctcaataaagcttgccttgagtgcttcaagtagtgtgtgcccgtctgttgtgtgactctggtaact | |
| | agagatccctcagacccttttagtcagtgtggaaaatctctagcagcatctagaattaattccgtgtattctatagtgtcac | |
| | ctaaatcgtatgtgtatgatacataaggttatgtattaattgtagccgcgttctaacgacaatatgtacaagcctaattgtgt | |
| | agcatctggcttactgaagcagaccctatcatctctctcgtaaactgccgtcagagtcggtttggttggacgaaccttct | |
| | gagtttctggtaacgccgtcccgcacccggaaatggtcagcgaaccaatcagcagggtcatcgctagccagatcctc | |
| | tacgccggacgcatcgtggccggcatcaccggcgccacaggtgcggttgctggcgcctatatcgccgacatcacc | |
| | gatggggaagatcgggctcgccacttcgggctcatgagcgcttgtttcggcgtgggtatggtggcaggccccgtgg | |
| | ccgggggactgttgggcgccatctccttgcatgcaccattccttgcggcggcggtgctcaacggcctcaacctacta | |
| | ctgggctgcttcctaatgcaggagtcgcataagggagagcgtcgaatggtgcactctcagtacaatctagctctgatg | |
| | ccgcatagttaagccagccccgacacccgccaacacccgctgacgcgccctgacgggcttgtctgctcccggcatc | |
| | cgcttacagacaagctgtgaccgtctccgggagctgcatgtgtcagaggttttcaccgtcatcaccgaaacgcgcga | |
| | gacgaaagggcctcgtgatacgcctatttttataggttaatgtcatgataataatggtttcttagacgtcaggtggcacttt | |
| | tcggggaaatgtgcgcggaacccctatttgtttatttttctaaatacattcaaatatgtatccgctcatgagacaataaccc | |
| | tgataaatgcttcaataatattgaaaaaggaagagtatgagtattcaacatttccgtgtcgcccttattcccttttttgcggc | |
| | attttgccttcctgtttttgctcacccagaaacgctggtgaaagtaaaagatgctgaagatcagttgggtgcacgagtgg | |
| | gttacatcgaactggatctcaacagcggtaagatccttgagagttttcgccccgaagaacgttttccaatgatgagcac | |
| | ttttaaagttctgctatgtggcgcggtattatcccgtattgacgccgggcaagagcaactcggtcgccgcatacactatt | |
| | ctcagaatgacttggttgagtactcaccagtcacagaaaagcatcttacggatggcatgacagtaagagaattatgca | |
| | gtgctgccataaccatgagtgataacactgcggccaacttacttctgacaacgatcggaggaccgaaggagctaacc | |
| | gcttttttgcacaacatgggggatcatgtaactcgccttgatcgttgggaaccggagctgaatgaagccataccaaac | |
| | gacgagcgtgacaccacgatgcctgtagcaatggcaacaacgttgcgcaaactattaactggcgaactacttactcta | |
| | gcttcccggcaacaattaatagactggatggaggcggataaagttgcaggaccacttctgcgctcggcccttccggc | |
| | tggctggtttattgctgataaatctggagccggtgagcgtgggtctcgcggtatcattgcagcactggggccagatgg | |
| | taagccctcccgtatcgtagttatctacacgacggggagtcaggcaactatggatgaacgaaatagacagatcgctg | |
| | agataggtgcctcactgattaagcattggtaactgtcagaccaagtttactcatatatactttagattgatttaaaacttcat | |
| | ttttaatttaaaaggatctaggtgaagatcctttttgataatctcatgaccaaaatcccttaacgtgagttttcgttccactga | |
| | gcgtcagaccccgtagaaaagatcaaaggatcttcttgagatcctttttttctgcgcgtaatctgctgcttgcaaacaaa | |
| | aaaaccaccgctaccagcggtggtttgtttgccggatcaagagctaccaactctttttccgaaggtaactggcttcagc | |
| | agagcgcagataccaaatactgtccttctagtgtagccgtagttaggccaccacttcaagaactctgtagcaccgccta | |
| | catacctcgctctgctaatcctgttaccagtggctgctgccagtggcgataagtcgtgtcttaccgggttggactcaag | |
| | acgatagttaccggataaggcgcagcggtcgggctgaacggggggttcgtgcacacagcccagcttggagcgaa | |
| | cgacctacaccgaactgagatacctacagcgtgagctatgagaaagcgccacgcttcccgaagggagaaaggcg | |
| | gacaggtatccggtaagcggcagggtcggaacaggagagcgcacgagggagcttccagggggaaacgcctggt | |
| | atctttatagtcctgtcgggtttcgccacctctgacttgagcgtcgatttttgtgatgctcgtcaggggggcggagcctat | |
| | ggaaaaacgccagcaacgcggcctttttacggttcctggccttttgctggccttttgctcacatgttctttcctgcgttatc | |
| | ccctgattctgtggataaccgtattaccgcctttgagtgagctgataccgctcgccgcagccgaacgaccgagcgca | |
| | gcgagtcagtgagcgaggaagcggaagagcgcccaatacgcaaaccgcctctccccgcgcgttggccgattcatt | |
| | aatgcagctgtggaatgtgtgtcagttagggtgtggaaagtccccaggctccccagcaggcagaagtatgcaaagc | |
| | atgcatctcaattagtcagcaaccaggtgtggaaagtccccaggctccccagcaggcagaagtatgcaaagcatgc | |
| | atctcaattagtcagcaaccatagtcccgcccctaactccgcccatcccgcccctaactccgcccagttccgcccattc | |
| | tccgccccatggctgactaattttttttatttatgcagaggccgaggccgcctcggcctctgagctattccagaagtagt | |
| | gaggaggcttttttggaggcctaggcttttgcaaaaagcttggacacaagacaggcttgcgagatatgtttgagaatac | |
| | cactttatcccgcgtcagggagaggcagtgcgtaaaaagacgcggactcatgtgaaatactggtttttagtgcgccag | |
| | atctctataatctcgcgcaacctattttcccctcgaacactttttaagccgtagataaacaggctgggacacttcacatga | |
| | gcgaaaaatacatcgtcacctgggacatgttgcagatccatgcacgtaaactcgcaagccgactgatgccttctgaac | |
| | aatggaaaggcattattgccgtaagccgtggcggtctgtaccgggtgcgttactggcgcgtgaactgggtattcgtca | |
| | tgtcgataccgtttgtatttccagctacgatcacgacaaccagcgcgagcttaaagtgctgaaacgcgcagaaggcg | |
| | atggcgaaggcttcatcgttattgatgacctggtggataccggtggtactgcggttgcgattcgtgaaatgtatccaaa | |
| | agcgcactttgtcaccatcttcgcaaaaccggctggtcgtccgctggttgatgactatgttgttgatatcccgcaagata | |
| | cctggattgaacagccgtgggatatgggcgtcgtattcgtcccgccaatctccggtcgctaatcttttcaacgcctggc | |
| | actgccgggcgttgttctttttaacttcaggcgggttacaatagtttccagtaagtattctggaggctgcatccatgacac | |
| | aggcaaacctgagcgaaaccctgttcaaaccccgctttaaacatcctgaaacctcgacgctagtccgccgctttaatc | |
| | acggcgcacaaccgcctgtgcagtcggcccttgatggtaaaaccatccctcactggtatcgcatgattaaccgtctga | |
| | tgtggatctggcgcggcattgacccacgcgaaatcctcgacgtccaggcacgtattgtgatgagcgatgccgaacgt | |
| | accgacgatgatttatacgatacggtgattggctaccgtggcggcaactggatttatgagtgggccccggatctttgtg | |
| | aaggaaccttacttctgtggtgtgacataattggacaaactacctacagagatttaaagctctaaggtaaatataaaattt | |
| | ttaagtgtataatgtgttaaactactgattctaattgtttgtgtattttagattccaacctatggaactgatgaatgggagcag | |
| | tggtggaatgcctttaatgaggaaaacctgttttgctcagaagaaatgccatctagtgatgatgaggctactgctgactc | |
| | tcaacattctactcctccaaaaaagaagagaaaggtagaagaccccaaggactttccttcagaattgctaagttttttga | |
| | gtcatgctgtgtttagtaatagaactcttgcttgctttgctatttacaccacaaaggaaaaagctgcactgctatacaaga | |
| | aaattatggaaaaatattctgtaacctttataagtaggcataacagttataatcataacatactgttttttcttactccacaca | |
| | ggcatagagtgtctgctattaataactatgctcaaaaattgtgtacctttagctttttaatttgtaaaggggttaataaggaa | |
| | tatttgatgtatagtgccttgactagagatcataatcagccataccacatttgtagaggttttacttgctttaaaaaacctcc | |
| | cacacctccccctgaacctgaaacataaaatgaatgcaattgttgttgttaacttgtttattgcagcttataatggttacaa | |
| | ataaagcaatagcatcacaaatttcacaaataaagcatttttttcactgcattctagttgtggtttgtccaaactcatcaatg | |
| | tatcttatcatgtctggatcaactggataactcaagctaaccaaaatcatcccaaacttcccaccccataccctattacca | |
| | ctgccaattacctagtggtttcatttactctaaacctgtgattcctctgaattattttcattttaaagaaattgtatttgttaaata | |
| | tgtactacaaacttagtagttggaagggctaattcactcccaaagaagacaagatatccttgatctgtggatctaccaca | |
| | cacaaggctacttccctgattagcagaactacacaccagggccaggggtcagatatccactgacctttggatggtgct | |
| | acaagctagtaccagttgagccagataaggtagaagaggccaataaaggagagaacaccagcttgttacaccctgt | |
| | gagcctgcatgggatggatgacccggagagagaagtgttagagtggaggtttgacagccgcctagcatttcatcac | |
| | gtggcccgagagctgcatccggagtacttcaagaactgctgatatcgagcttgctacaagggactttccgctgggga | |
| | ctttccagggaggcgtggcctggggggactggggagtggcgagccctcagatcctgcatataagcagctgcttttt | |
| | gcctgtactgggtctctctggttagaccagatctgagcctgggagctctctggctaactagggaacccactgcttaagc | |
| | ctcaataaagcttgccttgagtgcttcaagtagtgtgtgcccgtctgttgtgtgactctggtaactagagatccctcagac | |
| | ccttttagtcagtgtggaaaatctctagcagtggcgcccgaacagggacttgaaagcgaaagggaaaccagaggag | |
| | ctctctcgacgcaggactcggcttgctgaagcgcgcacggcaagaggcgaggggcggcgactggtgagtacgcc | |
| | aaaaattttgactagcggaggctagaaggagagagatgggtgcgagagcgtcagtattaagcgggggagaattag | |
| | atcgcgatgggaaaaaattcggttaaggccagggggaaagaaaaaatataaattaaaacatatagtatgggcaagca | |
| | gggagctagaactgattcgcagttaatcctggcctgttagaaacatcagaaggctgtagacaaatactgggacagcta | |
| | caaccatcccttcagacaggatcagaagaacttagatcattatataatacagtagcaaccctctattgtgtgcatcaaag | |
| | gatagagataaaagacaccaaggaagctttagacaagatagaggaagagcaaaacaaaagtaagaccaccgcac | |
| | agcaagcggccggtgatcttcagacctggacgatatatatgagggacaattggagaagtgaattatataaatataaag | |
| | tagtaaaaattgaaccattaggagtagcacccaccaaggcaaagagaagagtggtgcagagagaaaaaagagcag | |
| | tgggaataggagctttgttccttgggttcttgggagcagcaggaagcactatgggcgcagcgtcaatgacgctgacg | |
| | gtacaggccagacaattattgtctggtatagtgcagcagcagaacaatttgctgagggctattgaggcgcaacagcat | |
| | ctgttgcaactcacagtctggggcatcaagcagctccaggcaagaatcctggctgtggaaagatacctaaaggatca | |
| | acagctcctggggatttggggttgctctggaaaactcatttgcaccactgctgtgccttggaatgctagttggagtaata | |
| | aatctctggaacagatttggaatcacacgacctggatggagtgggacagagaaattaacaattacacaagcttaatac | |
| | actccttaattgaagaatcgcaaaaccagcaagaaaagaatgaacaagaattattggaattagataaatgggcaagttt | |
| | gtggaattggtttaacataacaaattggctgtggtatataaaattattcataatgatagtaggaggcttggtaggtttaaga | |
| | atagtttttgctgtactttctatagtgaatagagttaggcagggatattcaccattatcgtttcagacccacctcccaaccc | |
| | cgaggggacccgacaggcccgaaggaatagaagaagaaggtggagagagagacagagacagatccattcgatt | |
| | agtgaacggatctcgacggtcgccaaatggcagtattcatccacaattttaaaagaaaaggggggattggggggtac | |
| | agtgcaggggaaagaatagtagacataatagcaacagacatacaaactaaagaattacaaaaacaaattacaaaaat | |
| | tcaaaattttcgggtttattacagggacagcagagatccagtttggatcgataagcttgatatcgaatt | |
| 2 | gccgccaccatggccttaccagtgaccgccttgctcctgccgctggccttgctgctccacgccgccaggccggagc | Receptor GG0 |
| | agaagctgatcagcgaggaggacctggacatccagatgacacagactacatcctccctgtctgcctctctgggagac | sequence: Anti- |
| | agagtcaccatcagttgcagggcaagtcaggacattagtaaatatttaaattggtatcagcagaaaccagatggaact | CD19scFv-CD8Hinge- |
| | gttaaactcctgatctaccatacatcaagattacactcaggagtcccatcaaggttcagtggcagtgggtctggaacag | PolyValine TMD- |
| | attattctctcaccattagcaacctggagcaagaagatattgccacttacttttgccaacagggtaatacgcttccgtaca | Notch2JMD-Gal4 VP64 |
| | cgttcggaggggggaccaagctggagatcacaggtggcggtggctcgggcggtggtgggtcgggtggcggcgg | |
| | atctgaggtgaaactgcaggagtcaggacctggcctggtggcgccctcacagagcctgtccgtcacatgcactgtct | |
| | caggggtctcattacccgactatggtgtaagctggattcgccagcctccacgaaagggtctggagtggctgggagta | |
| | atatggggtagtgaaaccacatactataattcagctctcaaatccagactgaccatcatcaaggacaactccaagagc | |
| | caagttttcttaaaaatgaacagtctgcaaactgatgacacagccatttactactgtgccaaacattattactacggtggt | |
| | agctatgctatggactactggggccaaggaacctcagtcaccgtctcctcaaccacgacgccagcgccgcgaccac | |
| | caacaccggcgcccaccatcgcgtcgcagcccctgtccctgcgcccagaggcgtgcgttgtcgttgtggtggttgtg | |
| | gttgttgtcgtggtggttgtggtcgtggttgtggttgtcgtcaagaggaaacggaagcacatgaagctgctgagcagc | |
| | atcgagcaggcctgtgacatctgccggctgaagaaactgaagtgcagcaaagaaaagcccaagtgcgccaagtgc | |
| | ctgaagaacaactgggagtgccggtacagccccaagaccaagagaagccccctgaccagagcccacctgaccga | |
| | ggtggaaagccggctggaaagactggaacagctgtttctgctgatcttcccacgcgaggacctggacatgatcctga | |
| | agatggacagcctgcaggacatcaaggccctgctgaccggcctgttcgtgcaggacaacgtgaacaaggacgccg | |
| | tgaccgacagactggccagcgtggaaaccgacatgcccctgaccctgcggcagcacagaatcagcgccaccagc | |
| | agcagcgaggaaagcagcaacaagggccagcggcagctgacagtgtctgctgctgcaggcggaagcggaggct | |
| | ctggcggatctgatgccctggacgacttcgacctggatatgctgggcagcgacgccctggatgattttgatctggaca | |
| | tgctgggatctgacgctctggacgatttcgatctcgacatgttgggatcagatgcactggatgactttgacctggacat | |
| | gctcggatcatga | |
| 3 | gccgccaccatggccttaccagtgaccgccttgctcctgccgctggccttgctgctccacgccgccaggccggagc | GG1 sequence (Anti- |
| | agaagctgatcagcgaggaggacctggacatccagatgacacagactacatcctccctgtctgcctctctgggagac | CD19scFv- |
| | agagtcaccatcagttgcagggcaagtcaggacattagtaaatatttaaattggtatcagcagaaaccagatggaact | CD8αHinge- |
| | gttaaactcctgatctaccatacatcaagattacactcaggagtcccatcaaggttcagtggcagtgggtctggaacag | PolyValine TMD-GG1- |
| | attattctctcaccattagcaacctggagcaagaagatattgccacttacttttgccaacagggtaatacgcttccgtaca | Notch2JMD-Gal4 VP64 |
| | cgttcggaggggggaccaagctggagatcacaggtggcggtggctcgggcggtggtgggtcgggtggcggcgg | |
| | atctgaggtgaaactgcaggagtcaggacctggcctggtggcgccctcacagagcctgtccgtcacatgcactgtct | |
| | caggggtctcattacccgactatggtgtaagctggattcgccagcctccacgaaagggtctggagtggctgggagta | |
| | atatggggtagtgaaaccacatactataattcagctctcaaatccagactgaccatcatcaaggacaactccaagagc | |
| | caagttttcttaaaaatgaacagtctgcaaactgatgacacagccatttactactgtgccaaacattattactacggtggt | |
| | agctatgctatggactactggggccaaggaacctcagtcaccgtctcctcaaccacgacgccagcgccgcgaccac | |
| | caacaccggcgcccaccatcgcgtcgcagcccctgtccctgcgcccagaggcgtgcggtggcgttgtggtggttgt | |
| | ggttgttgtcgtggtggttgtggtcgtggttgtggttgtcgtcaagaggaaacggaagcacatgaagctgctgagcag | |
| | catcgagcaggcctgtgacatctgccggctgaagaaactgaagtgcagcaaagaaaagcccaagtgcgccaagtg | |
| | cctgaagaacaactgggagtgccggtacagccccaagaccaagagaagccccctgaccagagcccacctgaccg | |
| | aggtggaaagccggctggaaagactggaacagctgtttctgctgatcttcccacgcgaggacctggacatgatcctg | |
| | aagatggacagcctgcaggacatcaaggccctgctgaccggcctgttcgtgcaggacaacgtgaacaaggacgcc | |
| | gtgaccgacagactggccagcgtggaaaccgacatgcccctgaccctgcggcagcacagaatcagcgccaccag | |
| | cagcagcgaggaaagcagcaacaagggccagcggcagctgacagtgtctgctgctgcaggcggaagcggagg | |
| | ctctggcggatctgatgccctggacgacttcgacctggatatgctgggcagcgacgccctggatgattttgatctgga | |
| | catgctgggatctgacgctctggacgatttcgatctcgacatgttgggatcagatgcactggatgactttgacctggac | |
| | atgctcggatcatga | |
| 4 | gttggcggtgtggtggttgtggttgttgtcgtggtggttgtggtcgtggttgtggttgtcgtc | GG2 polyvaline |
| | | transmembrane domain |
| 5 | gttgtcggtggggtggttgtggttgttgtcgtggtggttgtggtcgtggttgtggttgtcgtc | GG3 polyvaline |
| | | transmembrane domain |
| 6 | gttgtcgttgggggggttgtggttgttgtcgtggtggttgtggtcgtggttgtggttgtcgtc | GG4 polyvaline |
| | | transmembrane domain |
| 7 | gttgtcgttgtggggggtgtggttgttgtcgtggtggttgtggtcgtggttgtggttgtcgtc | GG5 polyvaline |
| | | transmembrane domain |
| 8 | gttgtcgttgtggtgggtggggttgttgtcgtggtggttgtggtcgtggttgtggttgtcgtc | GG6 polyvaline |
| | | transmembrane domain |
| 9 | gttgtcgttgtggtggttgggggtgttgtcgtggtggttgtggtcgtggttgtggttgtcgtc | GG7 polyvaline |
| | | transmembrane domain |
| 10 | gttgtcgttgtggtggttgtgggtggtgtcgtggtggttgtggtcgtggttgtggttgtcgtc | GG8 polyvaline |
| | | transmembrane domain |
| 11 | gttgtcgttgtggtggttgtggttggtggcgtggtggttgtggtcgtggttgtggttgtcgtc | GG9 polyvaline |
| | | transmembrane domain |
| 12 | gttgtcgttgtggtggttgtggttgttggcggggtggttgtggtcgtggttgtggttgtcgtc | GG10 polyvaline |
| | | transmembrane domain |
| 13 | gttgtcgttgtggtggttgtggttgttgtcgggggggttgtggtcgtggttgtggttgtcgtc | GG11 polyvaline |
| | | transmembrane domain |
| 14 | gttgtcgttgtggtggttgtggttgttgtcgtggggggtgtggtcgtggttgtggttgtcgtc | GG12 polyvaline |
| | | transmembrane domain |
| 15 | gttgtcgttgtggtggttgtggttgttgtcgtggtgggtggggtcgtggttgtggttgtcgtc | GG13 polyvaline |
| | | transmembrane domain |
| 16 | gttgtcgttgtggtggttgtggttgttgtcgtggtggttgggggcgtggttgtggttgtcgtc | GG14 polyvaline |
| | | transmembrane domain |
| 17 | gttgtcgttgtggtggttgtggttgttgtcgtggtggttgtgggcggggttgtggttgtcgtc | GG15 polyvaline |
| | | transmembrane domain |
| 18 | gttgtcgttgtggtggttgtggttgttgtcgtggtggttgtggtcgggggtgtggttgtcgtc | GG16 polyvaline |
| | | transmembrane domain |
| 19 | gttgtcgttgtggtggttgtggttgttgtcgtggtggttgtggtcgtgggggggttgtcgtc | GG17 polyvaline |
| | | transmembrane domain |
| 20 | gttgtcgttgtggtggttgtggttgttgtcgtggtggttgtggtcgtggttgggggtgtcgtc | GG18 polyvaline |
| | | transmembrane domain |
| 21 | gttgtcgttgtggtggttgtggttgttgtcgtggtggttgtggtcgtggttgtgggtggcgtc | GG19 polyvaline |
| | | transmembrane domain |
| 22 | gttgtcgttgtggtggttgtggttgttgtcgtggtggttgtggtcgtggttgtggttggcggc | GG20 polyvaline |
| | | transmembrane domain |
| 23 | gccgccacc | Kozak sequence |
| 24 | atggccttaccagtgaccgccttgctcctgccgctggccttgctgctccacgccgccaggccg | CD8α signal peptide |
| 25 | gagcagaagctgatcagcgaggaggacctg | myc-tag |
| 26 | gacatccagatgacacagactacatcctccctgtctgcctctctgggagacagagtcaccatcagttgcagggcaagt | anti-CD19 scFv |
| | caggacattagtaaatatttaaattggtatcagcagaaaccagatggaactgttaaactcctgatctaccatacatcaag | |
| | attacactcaggagtcccatcaaggttcagtggcagtgggtctggaacagattattctctcaccattagcaacctggag | |
| | caagaagatattgccacttacttttgccaacagggtaatacgcttccgtacacgttcggaggggggaccaagctgga | |
| | gatcacaggtggcggtggctcgggcggtggtgggtcgggtggcggcggatctgaggtgaaactgcaggagtcag | |
| | gacctggcctggtggcgccctcacagagcctgtccgtcacatgcactgtctcaggggtctcattacccgactatggtg | |
| | taagctggattcgccagcctccacgaaagggtctggagtggctgggagtaatatggggtagtgaaaccacatactat | |
| | aattcagctctcaaatccagactgaccatcatcaaggacaactccaagagccaagttttcttaaaaatgaacagtctgc | |
| | aaactgatgacacagccatttactactgtgccaaacattattactacggtggtagctatgctatggactactggggccaa | |
| | ggaacctcagtcaccgtctcctca | |
| 27 | gttgtcgttgtggtggttgtggttgttgtcgtggtggttgtggtcgtggttgtggttgtcgtc | Polyvaline |
| | | transmembrane domain |
| 28 | aagaggaaacggaagcac | Notch2 juxtamembrane |
| | | domain |
| 29 | atgaagctgctgagcagcatcgagcaggcctgtgacatctgccggctgaagaaactgaagtgcagcaaagaaaag | Gal4-VP64 |
| | cccaagtgcgccaagtgcctgaagaacaactgggagtgccggtacagccccaagaccaagagaagccccctgac | |
| | cagagcccacctgaccgaggtggaaagccggctggaaagactggaacagctgtttctgctgatcttcccacgcgag | |
| | gacctggacatgatcctgaagatggacagcctgcaggacatcaaggccctgctgaccggcctgttcgtgcaggaca | |
| | acgtgaacaaggacgccgtgaccgacagactggccagcgtggaaaccgacatgcccctgaccctgcggcagcac | |
| | agaatcagcgccaccagcagcagcgaggaaagcagcaacaagggccagcggcagctgacagtgtctgctgctg | |
| | caggcggaagcggaggctctggcggatctgatgccctggacgacttcgacctggatatgctgggcagcgacgccc | |
| | tggatgattttgatctggacatgctgggatctgacgctctggacgatttcgatctcgacatgttgggatcagatgcactg | |
| | gatgactttgacctggacatgctcggatcatga | |
| 30 | ggtggcgttgtggtggttgtggttgttgtcgtggtggttgtggtcgtggttgtggttgtcgtc | Polyvaline |
| | | transmembrane domain |
| | | with GG in positions 1 |
| | | and 2 |
| 31 | MALPVTALLLPLALLLHAARP | CD8α signal peptide |
| 32 | EQKLISEEDL | myc-tag |
| 33 | GGAGCACTGTCCTCCGAACG | GAL4 DNA binding |
| | | domain target sequence |
| 34 | VVVVVVVVVVVVVVVVVVVVV | GG0 |
| 35 | GGVVVVVVVVVVVVVVVVVVV | GG1 |
| 36 | VGGVVVVVVVVVVVVVVVVVV | GG2 |
| 37 | VVVGGVVVVVVVVVVVVVVVV | GG3 |
| 38 | VVVGGVVVVVVVVVVVVVVVV | GG4 |
| 39 | VVVVGGVVVVVVVVVVVVVVV | GG5 |
| 40 | VVVVVGGVVVVVVVVVVVVVV | GG6 |
| 41 | VVVVVVGGVVVVVVVVVVVVV | GG7 |
| 42 | VVVVVVVGGVVVVVVVVVVVV | GG8 |
| 43 | VVVVVVVVGGVVVVVVVVVVV | GG9 |
| 44 | VVVVVVVVVGGVVVVVVVVVV | GG10 |
| 45 | VVVVVVVVVVGGVVVVVVVVV | GG11 |
| 46 | VVVVVVVVVVVGGVVVVVVVV | GG12 |
| 47 | VVVVVVVVVVVVGGVVVVVVV | GG13 |
| 48 | VVVVVVVVVVVVVGGVVVVVV | GG14 |
| 49 | VVVVVVVVVVVVVVGGVVVVV | GG15 |
| 50 | VVVVVVVVVVVVVVVGGVVVV | GG16 |
| 51 | VVVVVVVVVVVVVVVVGGVVV | GG17 |
| 52 | VVVVVVVVVVVVVVVVVGGVV | GG18 |
| 53 | VVVVVVVVVVVVVVVVVVGGV | GG19 |
| 54 | VVVVVVVVVVVVVVVVVVVGG | GG20 |
| 55 | accacgacgccagcgccgcgaccaccaacaccggcgcccaccatcgcgtcgcagcccctgt | CD8α-derived Hinge |
| | ccctgcgcccagaggcgtgc | Domain |
| 56 | TTTPAPRPPTPAPTIASQPLSLRPEAC | CD8α-derived Hinge |
| | | Domain |
| 57 | MKLLSSIEQACDICRLKKLKCSKEKPKCAKCLKNNWECRYSPKTKRSPLT | Gal4-VP64 |
| | RAHLTEVESRLERLEQLFLLIFPREDLDMILKMDSLQDIKALLTGLFVQDN | |
| | VNKDAVTDRLASVETDMPLTLRQHRISATSSSEESSNKGQRQLTVSAAAG | |
| | GSGGSGGSDALDDFDLDMLGSDALDDFDLDMLGSDALDDFDLDMLGSD | |
| | ALDDFDLDMLGS | |
| 58 | KRKRKH | Notch2 juxtamembrane |
| | | domain |
| 59 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 34 |
| | YGGSYAMDYWGQGTSVTVSSTTTPAPRPPTPAPTIASQPLSLRPEACVVV | |
| | VVVVVVVVVVVVVVVVVVKRKRKHMKLLSSIEQACDICRLKKLKCSKE | |
| | KPKCAKCLKNNWECRYSPKTKRSPLTRAHLTEVESRLERLEQLFLLIFPRE | |
| | DLDMILKMDSLQDIKALLTGLFVQDNVNKDAVTDRLASVETDMPLTLRQ | |
| | HRISATSSSEESSNKGQRQLTVSAAAGGSGGSGGSDALDDFDLDMLGSDA | |
| | LDDFDLDMLGSDALDDFDLDMLGSDALDDFDLDMLGS | |
| 60 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 35**: |
| | YGGSYAMDYWGQGTSVTVSSTTTPAPRPPTPAPTIASQPLSLRPEAC | |
| | GGVVVVVVVVVVVVVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 61 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 36**: |
| | YGGSYAMDYWGQGTSVTVSSTTTPAPRPPTPAPTIASQPLSLRPEAC | |
| | GGVVVVVVVVVVVVVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 62 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 37**: |
| | YGGSYAMDYWGQGTSVTVSSTTTPAPRPPTPAPTIASQPLSLRPEAC | |
| | VVGGVVVVVVVVVVVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 63 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 38**: |
| | YGGSYAMDYWGQGTSVTVSSTTTPAPRPPTPAPTIASQPLSLRPEAC | |
| | VVVGGVVVVVVVVVVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 64 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 39**: |
| | YGGSYAMDYWGQGTSVTVSSTTTPAPRPPTPAPTIASQPLSLRPEAC | |
| | VVVVGGVVVVVVVVVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 65 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 40**: |
| | YGGSYAMDYWGQGTSVTVSSTTTPAPRPPTPAPTIASQPLSLRPEAC | |
| | VVVVVGGVVVVVVVVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 66 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 41**: |
| | YGGSYAMDYWGQGTSVTVSSTTTPAPRPPTPAPTIASQPLSLRPEAC | |
| | VVVVVVGGVVVVVVVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 67 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 42**: |
| | YGGSYAMDYWGQGTSVTVSSTTTPAPRPPTPAPTIASQPLSLRPEAC | |
| | VVVVVVVGGVVVVVVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 68 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 43**: |
| | YGGSYAMDYWGQGTSVTVSSTTTPAPRPPTPAPTIASQPLSLRPEAC | |
| | VVVVVVVVGGVVVVVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 69 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 44**: |
| | YGGSYAMDYWGQGTSVTVSSTTTPAPRPPTPAPTIASQPLSLRPEAC | |
| | VVVVVVVVVGGVVVVVVVVVVKRKRKHMKLLSSIEQADIRLKKK | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 70 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 45**: |
| | YGGSYAMDYWGQGTSVTVSSTTTPAPRPPTPAPTIASQPLSLRPEAC | |
| | VVVVVVVVVVGGVVVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 71 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 46**: |
| | YGGSYAMDYWGQGTSVTVSSTTTPAPRPPTPAPTIASQPLSLRPEAC | |
| | VVVVVVVVVVVGGVVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 72 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 47**: |
| | YGGSYAMDYWGQGTSVTVSSTTTPAPRPPTPAPTIASQPLSLRPEAC | |
| | VVVVVVVVVVVVGGVVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 73 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 48**: |
| | YGGSYAMDYWGQGTSVTVSSTTTPAPRPPTPAPTIASQPLSLRPEAC | |
| | VVVVVVVVVVVVVGGVVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 74 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 49**: |
| | YGGSYAMDYWGQGTSVTVSSTTTPAPRPPTPAPTIASQPLSLRPEAC | |
| | VVVVVVVVVVVVVVGGVVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 75 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 50**: |
| | YGGSYAMDYWGQGTSVTVSSTTTPAPRPPTPAPTIASQPLSLRPEAC | |
| | VVVVVVVVVVVVVVVGGVVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 76 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 51**: |
| | YGGSYAMDYWGQGTSVTVSSTTTPAPRPPTPAPTIASQPLSLRPEAC | |
| | VVVVVVVVVVVVVVVVGGVVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 77 | MALPVTALLLPLALLLHAARPEQKLISEEDLDIQMTQTTSSLSASLGDRVT | \|nucleotide SEQ 02 and |
| | ISCRASQDISKYLNWYQQKPDGTVKLLIYHTSRLHSGVPSRFSGSGSGTDY | Amino acid sequence |
| | SLTISNLEQEDIATYFCQQGNTLPYTFGGGTKLEITGGGGSGGGGSGGGGS | corresponding to |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 52**: |
| | YGGSYAMDYWGQGTSVTVSSTTTPAPRPPTPAPTIASQPLSLRPEAC | |
| | VVVVVVVVVVVVVVVVVGGVVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 78 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 53**: |
| | YGGSYAMDYWGQGTSVTVSSTTTPAPRPPTPAPTIASQPLSLRPEAC | |
| | VVVVVVVVVVVVVVVVVVGGVKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |
| 79 | MALPVTALLLPLALLLHAARPEQKLISEEDLDIQMTQTTSSLSASLGDRVT | Amino acid sequence |
| | ISCRASQDISKYLNWYQQKPDGTVKLLIYHTSRLHSGVPSRFSGSGSGTDY | corresponding to |
| | SLTISNLEQEDIATYFCQQGNTLPYTFGGGTKLEITGGGGSGGGGSGGGGS | nucleotide SEQ 02 and |
| | EVKLQESGPGLVAPSQSLSVTCTVSGVSLPDYGVSWIRQPPRKGLEWLGV | receptor including SEQ |
| | IWGSETTYYNSALKSRLTIIKDNSKSQVFLKMNSLQTDDTAIYYCAKHYY | ID 54**: |
| | YGGSYAMDYWGQGTSVTVSSTTTPAPRPPTPAPTIASQPLSLRPEAC | |
| | VVVVVVVVVVVVVVVVVVVGGKRKRKHMKLLSSIEQACDICRLKKLKC | |
| | SKEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDMLG | |
| | SDALDDFDLDMLGSDALDDFDLDMLGSDALDDFDLDMLGS | |

*Receptors GG1 to GG20 (GGX) are the same as GG0, except with a GG substitution at the numbered location X and X + 1 within the poly-valine TMD.

### REFERENCES

David L. Porter, M.D., Bruce L. Levine, Ph.D., Michael Kalos, Ph.D., Adam Bagg, M.D., and Carl H. June, M.D. Chimeric Antigen Receptor-Modified T Cells in Chronic Lymphoid Leukemia. N. Engl J Med. (2011) August 25; 365 (8): 725-33.
Gordon W R et al., The molecular logic of Notch signaling—a structural and biochemical perspective. J. Cell Sci. (2008) 121:3109-19.
Gordon W R et al., Mechanical Allostery: Evidence for a Force Requirement in the Proteolytic Activation of Notch. Dev Cell (2015) 33:729-36.
Haapasalo Al, Kovacs D M. The many substrates of presenilin/γ-secretase. J Alzheimers Dis. (2011); 25 (1): 3-28.
Rosmalen M., Krom M., and Merkx M. Tuning the Flexibility of Glycine-Serine Linkers To Allow Rational. Biochemistry (2017) 56:6565-6574.
Morsut L, Roybal K T, Xiong X, Gordley R M, Coyle S M, Thomson M, and Lim W A. Engineering Customized Cell Sensing and Response Behaviors Using Synthetic Notch Receptors. Cell. (2016) February 11; 164 (4): 780-91.
Naso M F, Tomkowicz B, Perry W L 3rd, Strohl W R. Adeno-Associated Virus (AAV) as a Vector for Gene Therapy. BioDrugs. (2017); 31 (4): 317-34.
Nasri M, Karimi A, Allahbakhshian Farsani M. Production, purification and titration of a lentivirus-based vector for gene delivery purposes. Cytotechnology. (2014); 66 (6): 1031-38.
Porter D L, Levine B L, Kalos M., Bagg A and June C H. Chimeric Antigen Receptor-Modified T Cells in Chronic Lymphoid Leukemia. Aug. 25, 2011. N. Engl. J. Med. (2011); 365:725-733.
Roybal K T, Jasper Z. Williams, Leonardo Morsut, Levi J. Rupp, Isabel Kolinko, Joseph H. Choe, Whitney J. Walker, Krista A. McNally, and Wendell A. Lim. Engineering T cells with Customized Therapeutic Response Programs Using Synthetic Notch Receptors. Cell. (2016) October 6; 167 (2): 419-32.
Samulski and Muzyczka. AAV-Mediated Gene Therapy for Research and Therapeutic Purposes. Annu. Rev. Virol. (2014) 1:427.
Sakuma, et al. (2012). Lentiviral vectors: basic to translational. Biochem. J. 443:603.
Watson D. J., Wolfe J. H. Viral vectors for gene therapy: methods and protocols. Totowa, NJ, USA: Humana Press; (2003). pp. 383-404.
Vidarsson G. et al., IgG subclasses and allotypes: from structure to effector functions. Frontiers Immunol. (2014) October 20; 5:520.
Zhang et al., The γ-secretase complex: from structure to function. Frontiers in Cellular Neuroscience. (2014) December 8: 427.

What is claimed is:

1. A chimeric polypeptide comprising:
(a) an extracellular ligand-binding domain (ECD) having a binding affinity for a selected ligand;
(b) a synthetic transmembrane domain (STMD) comprising one or more ligand-inducible proteolytic cleavage site; and
(c) an intracellular domain (ICD) comprising a transcriptional regulator (TR),
wherein binding of the selected ligand to the extracellular ligand-binding domain induces cleavage at the ligand-inducible proteolytic cleavage site and release of the transcriptional regulator.

2. The chimeric polypeptide of claim 1, wherein the chimeric polypeptide further comprises a hinge domain incorporated between the extracellular ligand-binding domain and the STMD.

3. The chimeric polypeptide of claim 1, wherein the chimeric polypeptide comprises the ECD, the STMD, and the ICD in order from N-terminus to C-terminus of the first polypeptide.

4. The chimeric polypeptide of claim 2, wherein the chimeric polypeptide comprises the ECD, the hinge domain, the STMD, and the ICD in order from N-terminus to C-terminus of the first polypeptide

5. The chimeric polypeptide of any one of claims 1 to 4, wherein the STMD comprises one or more valine residues.

6. The chimeric polypeptide of any one of claims 1 to 5, wherein the STMD comprises a series of at least 5 valine residues.

7. The chimeric polypeptide of any one of claims 1 to 6, wherein the STMD comprises between 5 to 30 valine residues.

8. The chimeric polypeptide of any one of claims 1 to 7, wherein the STMD further comprises two consecutive glycine residues.

9. The chimeric polypeptide of claim 8, the two consecutive glycine residues are located at any one of positions 5 to 30 of STMD, wherein position 30 is closer to the C-terminus of the chimeric polypeptide than position 5.

10. The chimeric polypeptide of any one of claims 1 to 9, wherein the STMD consists of valine residues.

11. The chimeric polypeptide of any one of claims 1 to 10, wherein the ligand comprises a protein or a carbohydrate.

12. The chimeric polypeptide of any one of claims 1 to 11, wherein the ligand is selected from cell surface receptors, adhesion proteins, integrins, mucins, lectins, tumor associated antigens, and tumor-specific antigens.

13. The chimeric polypeptide of any one of claims 1 to 12, wherein the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B-cell maturation antigen (BCMA), green fluorescent protein (GFP), enhanced green fluorescent Protein (eGFP), and signal regulatory protein α (SIRPα).

14. The chimeric polypeptide of any one of claims 11 to 13, wherein the extracellular binding domain comprises the ligand-binding portion of a receptor.

15. The chimeric polypeptide of any one of claims 1 to 14, wherein the ECD comprises an antigen-binding moiety capable of binding to a ligand on the surface of a cell.

16. The chimeric polypeptide of claim 15, wherein the antigen-binding moiety is selected from the group consisting of an antibody, a nanobody, a diabody, a triabody, or a minibody, a F(ab′)2 fragment, a Fab fragment, a single chain variable fragment (scFv), and a single domain antibody (sdAb), or a functional fragment thereof.

17. The chimeric polypeptide of claim 16, wherein the antigen-binding moiety comprises a scFv.

18. The chimeric polypeptide of any one of claims 1 to 17, wherein the antigen-binding moiety is capable of binding a tumor-associated antigen selected from the group consisting of CD19, B7H3 (CD276), BCMA, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRVIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD 117), MUC1, NCAM, PAP, PDGFR-beta, PRSS21, PSCA, PSMA, ROR1, SSEA-4, TAG72, TEM1/CD248, TEM7R, TSHR, VEGFR2, BCMA (CD269), ALPI, citrullinated vimentin, cMet, and Axl.

19. The chimeric polypeptide of claim 10, wherein the tumor-associated antigen is CD19, CEA, HER2, MUC1, CD20, or EGFR.

20. The chimeric polypeptide of claim 11, wherein the tumor-associated antigen is CD19.

21. The chimeric polypeptide of claim 15, wherein the cell is a pathogen.

22. The chimeric polypeptide of any one of claims 8 to 13, wherein the ligand-inducible proteolytic cleavage site is the two consecutive glycine residues.

23. The chimeric polypeptide of any one of claims 1 to 22, wherein the transcriptional regulator comprises a transcriptional activator, a transcriptional repressor, a site-specific nuclease, an inhibitory immunoreceptor, or an activating immunoreceptor.

24. The chimeric polypeptide of any one of claims 1 to 23, wherein the transcriptional regulator is selected from the group consisting of Gal4-VP16, Gal4-VP64, tetR-VP64, ZFHD1-VP64, Gal4-KRAB, and HAP1-VP16.

25. The chimeric polypeptide of any one of claims 1 to 23, further comprising a ligand-induced proteolytic cleavage site, a tumor-specific cleavage site, a disease-specific cleavage site, an autoproteolytic peptide sequence, a nuclear localization signal, a juxtamembrane domain, a signaling domain, or a combination of any combination thereof.

26. The chimeric polypeptide of claim 25, wherein the signaling domain is from DAP12, CD3 zeta, TCR zeta, FcR gamma, FcR beta, CD3 gamma, CD3 delta, CD3 epsilon, CD5, CD22, CD79a, CD79b, CD278 (ICOS), Fc.epsilon.RI, DAP10, DAP12, or CD66d.

27. The chimeric polypeptide of claim 25, wherein the juxtamembrane domain is a Notch 2 juxtamembrane domain or similar polybasic domain.

28. The chimeric polypeptide of claim 25, wherein the proteolytic cleavage site is cleavable by gamma secretase.

29. The chimeric polypeptide of claim 25, wherein the autoproteolytic peptide sequence is from a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

30. The chimeric polypeptide of any one of claims 2 to 25, wherein the hinge domain is from CD8α or CD28.

31. The chimeric polypeptide of claim 30, wherein the hinge domain is a truncated CD8α hinge domain.

32. The chimeric polypeptide of any one of claims 1 to 31, wherein the STMD comprises an amino acid sequence encoded by a nucleic acid sequence having at least 80%, 85%, 90%, 95%, 96%, 97%, 98%, 99% or 100% sequence identity to any one of SEQ ID NO: 2 to SEQ ID NO: 22, SEQ ID NO: 27, or SEQ ID NO: 30.

33. The chimeric polypeptide of any one of claims 1 to 32, wherein the STMD comprises an amino acid sequence having at least 80%, 85%, 90%, 95%, 96%, 97%, 98%, 99% or 100% sequence identity to any one of SEQ ID NO: 34, SEQ ID NO: 35, SEQ ID NO: 36, SEQ ID NO: 37, and SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO:50, SEQ ID NO: 51, SEQ ID NO: 52, SEQ ID NO: 53, SEQ ID NO: 54, and SEQ ID NO: 55.

34. The chimeric polypeptide of any one of claims 1 to 17, wherein the chimeric polypeptide is encoded by a nucleic acid sequence having at least 80%, 85%, 90%, 95%, 98%, or 99% sequence identity to SEQ ID NO: 2 or SEQ ID NO:3.

35. A STMD for a chimeric polypeptide according to any one of claims 1 to 34, wherein the STMD comprises at least 5 valine residues.

36. The STMD of claim 35, wherein the STMD comprises at least 10 valine residues.

37. The STMD of claim 35, wherein the STMD comprises at least 20 valine residues.

38. The STMD of claim 35, wherein the STMD comprises between 15 and 25 valine residues.

39. The STMD of any one of claims 35 to 38, wherein the STMD comprises a ligand-inducible proteolytic cleavage site and wherein binding of the selected ligand to the extracellular ligand-binding domain induces cleavage at the ligand-inducible proteolytic cleavage site and release of the transcriptional regulator.

40. A recombinant nucleic acid molecule comprising a nucleotide sequence encoding a chimeric polypeptide according to any one of claims 1 to 34.

41. The recombinant nucleic acid molecule of claim 40, wherein the recombinant nucleic acid molecule comprises a sequence having at least 80%, 85%, 90%, 95%, 98%, or 99% sequence identity to any one of SEQ ID NO: 2, SEQ ID NO: 22, SEQ ID NO: 27, and SEQ ID NO: 30.

42. A vector comprising the recombinant nucleic acid molecule of claim 40 or 41

43. The vector of claim 42, wherein the vector is an expression vector.

44. The vector of claim 43, wherein the expression vector is a viral vector.

45. The vector of claim 44, wherein the viral vector is a lentiviral vector, an adenovirus vector, an adeno-associated virus vector, or a retroviral vector.

46. A recombinant cell comprising:
a chimeric polypeptide according to any one of claims 1 to 34; or
a recombinant nucleic acid according to any one of claims 40 to 41; or
a vector according to any one of claims 42 to 45; or
a STMD according to any one of claims 35 to 39.

47. The recombinant cell of claim 46, wherein the cell is a mammalian cell.

48. The recombinant cell of claim 54, wherein the mammalian cell is an immune cell, a neuron, an epithelial cell, and endothelial cell, or a stem cell.

49. The recombinant cell of claim 48, wherein the immune cell is a B cell, a monocyte, a natural killer cell, a basophil, an eosinophil, a neutrophil, a dendritic cell, a macrophage, a regulatory T cell, a helper T cell, a cytotoxic T cell, a CD4+ T cell, a CD8+ T cell, or another T cell.

50. The recombinant cell of any one of claims 46 to 49 comprising the chimeric polypeptide according to any one of claims 1 to 34, wherein the recombinant cell further comprises a nucleic acid sequence encoding a protein operably linked to a promoter, wherein expression of the protein is modulated by the transcriptional regulator of the chimeric polypeptide.

51. The recombinant cell of claim 50, wherein the protein is heterologous.

52. The recombinant cell of claim 50 or 51, wherein the protein is a cytokine, a cytotoxin, a chemokine, an immunomodulator, a pro-apoptotic factor, an anti-apoptotic factor, a hormone, a differentiation factor, or a dedifferentiation factor.

53. A pharmaceutical composition comprising a recombinant cell according to any one of claims 46 to 52.

54. A method for modulating an activity of a cell, the method comprising:
providing a recombinant cell according to any one of claims 46 to 52 comprising the chimeric polypeptide according to any one of claims 1 to 34; and
contacting the recombinant cell with the selected ligand, wherein binding of the selected ligand to the extracellular binding domain induces cleavage of a ligand-inducible proteolytic cleavage site and releases the transcriptional regulator, wherein the released transcriptional regulator modulates an activity of the recombinant cell.

55. The method of claim 54, the contacting is carried out in vivo, ex vivo, or in vitro.

56. The method of any one of claim 54 or 55, wherein the activity of the cell is selected from the group consisting of: expression of a selected gene of the cell, proliferation of the cell, apoptosis of the cell, non-apoptotic death of the cell, differentiation of the cell, dedifferentiation of the cell, migration of the cell, secretion of a molecule from the cell, cellular adhesion of the cell, and cytolytic activity of the cell.

57. The method of any one of claims 54 to 56, wherein the released transcriptional regulator modulates expression of a gene product of the cell.

58. The method of claim 57, wherein the released transcriptional regulator modulates expression of a heterologous gene product.

59. The method of claim 57 or 58, wherein the gene product of the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, an RNA guided nuclease, a site-specific nuclease, a T cell receptor, a toxin, a toxin derived protein, a transcriptional activator, a transcriptional repressor, a translation regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

60. The method of any one of claims 54 to 59, wherein the released transcriptional regulator modulates differentiation of the cell, and wherein the cell is an immune cell, a stem cell, a progenitor cell, or a precursor cell.

61. The method of any one of claims 54 to 59, wherein the administered recombinant cell modulates an activity of a target cell in the individual.

62. The method of claim 61, wherein the target cell is a cancer cell.

63. The method of claim 62, wherein the cancer cell is a solid tumor or a hematological malignancy cell.

64. The method of claim 63, wherein the hematological malignancy cell is a multiple myeloma cell.

65. A method for the treatment of a health condition in an individual in need thereof, the method comprising: administering to the individual a first therapy comprising an effective number of the recombinant cell according to any one of claims 46 to 52, wherein the recombinant cell treats the disease in the individual.

66. The method of claim 65, wherein the disease is cancer.

67. The method of claim 66, wherein the cancer is a solid tumor.

68. The method claim 66, wherein the cancer is a hematological malignancy.

69. The method of claim 68, wherein the hematological malignancy is multiple myeloma.

70. The method of claim 69, further comprising administering to the individual a second therapy.

71. The method of claim 70, wherein the second therapy is selected from the group consisting of chemotherapy, radiotherapy, immunotherapy, hormonal therapy, and toxin therapy.

72. The method of any one of claims 65 to 71, wherein the first therapy and the second therapy are administered together, in the same composition or in separate compositions.

73. The method claim 72, wherein the first therapy and the second therapy are administered concomitantly.

74. The method of any one of claims 70 to 73, wherein the first therapy and the second therapy are administered sequentially.

75. The method of claim 72, wherein the first therapy is administered before the second therapy.

76. A method for inducing T cell signaling and gene regulation in a T cell comprising:
(a) providing a vector comprising the chimeric polypeptide according to any one of claims 1-41; and
(b) transducing a T cell with the vector,
wherein binding of a selected ligand to the extracellular ligand-binding domain of the chimeric polypeptide induces intracellular signaling and release of the transcriptional regulator.

77. A system for modulating an activity of a cell, killing a target cancer cell, or treating a disease in an individual in need thereof, wherein the system comprises one or more of the following:
a chimeric polypeptide according to any one of claims 1 to 34;
a recombinant nucleic acid molecule according to any one of claims 40 to 41;
a recombinant cell according to any one of claims 46 to 52; and
a pharmaceutical composition according to claim 53.

78. A method for making the recombinant cell according to any one of claims 46 to 52 comprising:
providing a cell capable of protein expression; and
contacting the provided cell with a recombinant nucleic acid molecule according to any one of claims 40 to 41.

79. The use of one or more of the following for the treatment of a disease:
a chimeric polypeptide according to any one of claims 1 to 34;
a recombinant nucleic acid molecule according to any one of claims 40 to 41; and
a recombinant cell according to any one of claims 46 to 52.

80. The use of claim 79, wherein the disease is cancer.

81. The use of the invention of any one of claims 1 to 53, for the manufacture of a medicament for the treatment of a disease.
